(19) United States
(12) Patent Application Publication
(10) Pub. No.: US 2022/0357346 A1
(43) Pub. Date: Nov. 10, 2022

(54) METHODS AND COMPOSITIONS FOR THE DETECTION, DIAGNOSIS AND TREATMENT OF AUTISM SPECTRUM DISORDER

(71) Applicants: The Johnson Center for Child Health and Development, Austin, TX (US); The Board of Regents of the University of Texas System, Austin, TX (US)

(72) Inventors: Laura C. Hollenbeck, Austin, TX (US); Dwight C. German, Dallas, TX (US); Jeon Lee, Carrollton, TX (US); Jeremy A. Mathews, Austin, TX (US)

(21) Appl. No.: 17/730,025

(22) Filed: Apr. 26, 2022

Related U.S. Application Data
(60) Provisional application No. 63/235,629, filed on Aug. 20, 2021.
(60) Provisional application No. 63/184,670, filed on May 5, 2021.

(51) Int. Cl. G01N 33/68

(52) U.S. Cl. CPC G01N 33/6896; G01N 2333/912

(57) ABSTRACT

Disclosures herein are directed to methods and compositions for the detection of a panel of proteins as markers for ASD. Based on the results achieved from the methods disclosed herein, ASD can be diagnosed and suitable treatments for ASD may be designed and administered to the subject.

### CROSS-REFERENCE TO RELATED APPLICATIONS

The present application claims priority to U.S. Provisional Patent Application 63/184,670, filed May 5, 2021, and U.S. Provisional Patent Application 63/235,629, Filed Aug. 20, 2021. Both applications are specifically incorporated herein in their entirety.

### FIELD OF THE INVENTION

The present inventive concept is directed to methods and compositions for the detection, diagnosis and/or treatment of Autism spectrum disorder (ASD) in a subject.

### BACKGROUND OF THE INVENTION

ASD is a neurodevelopmental disorder characterized by deficits in social communication and social interaction, and restricted, repetitive patterns of behavior, interests or activities. Early recognition of ASD is crucial because an early diagnosis can provide access to behavioral and educational therapies, which may reduce symptoms and support improved outcomes. Unfortunately, behavioral and developmental assessments are not easily administered in very young children. As such, there is a need in the field to identify novel diagnostic biomarkers with diagnostic accuracy for the identification of ASD, particularly in young subjects.

### SUMMARY OF THE INVENTION

The present disclosure is based, at least in part, on the on the identification of a panel of proteins as markers for ASD. Accordingly, provided herein are methods and kits for detecting and diagnosing ASD in a subject that rely on protein expression levels of a panel of proteins disclosed herein as markers for ASD. Based on the results achieved from the methods disclosed herein, suitable treatments for ASD may be designed and administered to the subject.

Aspects of the present disclosure provide for methods of diagnosing ASD in a subject. In some embodiments, methods disclosed herein can have one or more of the following steps: (a) obtaining a biological sample from the subject; (b) determining the level of at least one marker of ASD, wherein the at least one marker of ASD is selected from immunoglobulin D (IgD), soluble urokinase-type plasminogen activator receptor (suPAR), mitogen-activated protein kinase 14 (MAPK14), ephrin type-B receptor 2 (EPHB2), dermatopontin (DERM), and any combination thereof; and, (c) diagnosing the subject as having ASD if the level of at least one of IgD, suPAR, MAPK14, EPHB2, or DERM are differentially expressed as compared to a control, wherein the control can be a biological sample obtained from a subject diagnosed as not having ASD. In some embodiments, the at least one marker of ASD can further include at least one of receptor tyrosine kinase-like orphan receptor 1 (ROR1), platelet receptor Gl24 (GI24), eukaryotic translation initiation factor 4H (elF-4H), arylsulfatase B (ARSB), or any combination thereof. In some embodiments, the level of at least one marker of ASD can be determined by measuring the protein expression level of the at least one marker of ASD. In some embodiments, a subject herein may be diagnosed as having ASD if the levels of at least one of MAPK14, IgD, DERM, EPHB2, eIF-4H, and/or ROR1 are decreased as compared to the control and at least one of suPAR, ARSB and/or GI24 are increased as compared to the control.

In some embodiments, methods herein can further include administering to a subject at least one treatment for ASD. In some embodiments, the at least one treatment for ASD can be applied behavior analysis, assistive technology, social skills training, speech therapy, dietary treatment, pharmaceutical treatment, or any combination thereof.

In some embodiments, methods disclosed herein of diagnosing ASD in a subject can include a human patient as the subject. In some examples, a human patient suitable for methods herein can be not suspected of having, suspected of having, or having ASD. In some examples, a human patient suitable for methods herein can be a human child. In some examples, a human patient suitable for methods herein can be a human male child.

In some embodiments, methods disclosed herein can have a biological sample obtained from a subject wherein the biological sample can be a whole blood sample, a blood serum sample, a blood plasma sample, or any combination thereof.

Other aspects of the present disclosure provide for methods of diagnosing ASD in a subject wherein the methods can have one or more of the following steps: (a) obtaining a biological sample from the subject; (b) determining the levels of at least two markers of ASD, wherein (i) at least one of the at least two markers of ASD is selected from immunoglobulin D (IgD), soluble urokinase-type plasminogen activator receptor (suPAR), mitogen-activated protein kinase 14 (MAPK14), ephrin type-B receptor 2 (EPHB2), and dermatopontin (DERM); and (ii) at least one of the at least two markers of ASD is selected from receptor tyrosine kinase-like orphan receptor 1 (ROR1), platelet receptor Gl24 (GI24), eukaryotic translation initiation factor 4H (elF-4H), and arylsulfatase B (ARSB); and, (c) diagnosing the subject as having ASD if the levels of at least one of IgD, suPAR, MAPK14, EPHB2, DERM, or any combination thereof and at least one of ROR1, GI24, eIF-4H, ARSB, or any combination thereof are differentially expressed as compared to a control, wherein the control can be a biological sample obtained from a subject diagnosed as not having ASD.

In some embodiments, methods herein can diagnose a subject as having ASD if the levels of IgD, suPAR, MAPK14, EPHB2, and DERM are differentially expressed as compared to a control. In some embodiments, methods herein can diagnose a subject as having ASD if the levels of IgD, suPAR, MAPK14, EPHB2, and DERM are differentially expressed as compared to a control and at least one ROR1, Gl24, elF-4H, ARSB, or any combination thereof is differentially expressed as compared to a control. In some embodiments, methods herein can diagnose a subject as having ASD if the levels of IgD, suPAR, MAPK14, EPHB2, DERM, ROR1, Gl24, elF-4H, and ARSB are differentially expressed as compared to a control. In some embodiments, methods herein can diagnose a subject as having ASD if the levels of at least one of IgD, MAPK14, EPHB2, and/or DERM and at least one of ROR1 and/or eIF-4H are decreased as compared to the control and at least one of suPAR, ARSB and/or GI24 are increased as compared to the control. In some embodiments, methods herein can diagnose a subject as having ASD if the levels of IgD, MAPK14, EPHB2, and DERM are decreased as compared to a control and the levels of suPAR are increased as compared to a control. In some embodiments, methods herein can diagnose a subject as having ASD if the levels of IgD, MAPK14, EPHB2, and DERM and at least one of ROR1 and/or eIF-4H are decreased as compared to the control and at least one of suPAR, ARSB and/or GI24 are increased as compared to the control. In some embodiments, methods herein can diagnose a subject as having ASD if the levels of IgD, MAPK14, EPHB2, and DERM and at least one of ROR1 and/or eIF-4H are decreased as compared to a control and the levels of suPAR are increased as compared to a control. In some embodiments, methods herein can diagnose a subject as having ASD if the levels of IgD, MAPK14, EPHB2, DERM, ROR1 and eIF-4H are decreased as compared to the control and at least one of suPAR, ARSB and/or GI24 are increased as compared to the control. In some embodiments, methods herein can diagnose a subject as having ASD if the levels of at least one IgD, MAPK14, EPHB2, DERM, ROR1 and/or eIF-4H are decreased as compared to the control and suPAR, ARSB and GI24 are increased as compared to the control. In some embodiments, methods herein can diagnose a subject as having ASD if the levels of IgD, MAPK14, EPHB2, DERM, ROR1 and eIF-4H are decreased as compared to the control and suPAR, ARSB and GI24 are increased as compared to the control.

In various embodiments, methods herein can further include determining one or more non-genetic factors of ASD in the subject. In some examples, one or more non-genetic factors of ASD can be Autism Diagnostic Observation Schedule (ADOS), Autism Diagnostic Interview-Revised (ADI-R), the Adaptive Behavior Assessment System-Second Edition (ABAS-II), Diagnostic and Statistical Manual of Mental Disorders (DSM-5) Autism Diagnostic Criteria, or any combination thereof. In some examples, methods herein can include determining one or more non-genetic factors of ASD in the subject before determining the levels of at least one marker of ASD, after determining the levels of at least one marker of ADS, or while determining the levels of at least one marker of ASD.

Aspects of the present disclosure provide for methods of treating Autism spectrum disorder (ASD) in a subject. In some embodiments, methods of treating ASD include at least one of the disclosed methods for diagnosing ASD in a subject. In some embodiments, methods of treating ASD include at least one of the disclosed methods for diagnosing ASD in a subject and administering to the subject diagnosed as having ASD at least one treatment for ASD. In some examples, the at least one treatment for ASD can include applied behavior analysis, assistive technology, social skills training, speech therapy, dietary treatment, pharmaceutical treatment, or any combination thereof.

Other aspects of the present disclosure provide for kits for practicing any of the methods disclosed herein. In some embodiments, kits herein can at least have a means for determining protein levels of any combination of markers of ASD. In some examples, the combination of markers of ASD can be selected from the group of immunoglobulin D (IgD), soluble urokinase-type plasminogen activator receptor (suPAR), mitogen-activated protein kinase 14 (MAPK14), ephrin type-B receptor 2 (EPHB2), dermatopontin (DERM), receptor tyrosine kinase-like orphan receptor 1 (ROR1), platelet receptor Gl24 (GI24), eukaryotic translation initiation factor 4H (elF-4H), arylsulfatase B (ARSB), and any combination thereof In some embodiments, kits herein can have a set of aptamers, each of which for detecting one of the ASD markers in combination. In some examples, kits herein having a set of aptamers can collectively detect the protein levels of the markers in the combination. In some examples, kits herein can have a set of aptamers attached to a solid support. In some embodiments, kits herein can further include one or more of the following: (i) a tool for collecting a biological sample from the subject; (ii) a container for placing the biological sample; and/or (iii) one or more reagents for determining protein levels of the combination of markers of ASD from the biological sample.

### DESCRIPTION OF THE SEVERAL VIEWS OF THE DRAWINGS

Embodiments The patent or application file contains at least one drawing executed in color. Copies of this patent or patent application publication with colored drawings will be provided by the Office upon request and payment of the necessary fee.

Embodiments of the present inventive concept are illustrated by way of example in which like reference numerals indicate similar elements and in which:

FIG. 1 depicts an image illustrating a graph showing a range of ADOS scores among ASD subjects in accordance with certain aspects of the present disclosure where each dot represents the ADOS total score for a single subject.

FIGS. 2A-2C depict images illustrating the top predictive proteins for ASD as identified by three different methods in accordance with certain aspects of the present disclosure. FIG. 2A shows a graph depicting proteins identified by the Random Forest based method. FIG. 2B shows a volcano plot depicting proteins identified by the T-test based method where a total of 86 proteins were downregulated in the ASD subjects and 52 proteins were upregulated. All of these proteins had a false discovery rate<0.05. FIG. 2C shows a graph depicting proteins identified by the correlation based method.

FIGS. 3A-3C depict images illustrating optimization of the predictive proteins for ASD in accordance with certain aspects of the present disclosure. FIG. 3A shows a diagram depicting the identification of 5 core proteins where the core proteins were among the top-10 proteins identified by each of the 3 methods. FIG. 3B shows an image depicting identification of proteins with additive predictive power when combined with the core proteins. FIG. 3C shows an image depicting the predictive power (AUC) of the top-10 proteins from the three different methods: “RF”—random forest, “Ttest”—t-tests, and “Corr”—correlation compared with the optimal panel of 9 proteins (AUC Optimal) representing the 5 core proteins and the 4 additional proteins identified in FIG. 3B.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION OF THE INVENTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, at least in part, on the on the identification of markers for ASD—specifically, a panel of proteins as markers for ASD. Accordingly, the present disclosure provides for methods of detecting and/or diagnosing ASD in a subject, methods of treating ASD in a subject, and kits used in practicing the methods disclosed herein.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, the term “about” as used in the description and the appended claims, should be understood to include the recited values or a value that is three times greater or one third of the recited values. For example, about 3 cm includes all values from 1 mm to 9 cm. For example, terms of degree can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The term “solid support” as used herein refers to a material having a rigid or semi-rigid surface. Such materials will preferably take the form of small beads, pellets, disks, chips, or wafers, although other forms may be used.

The term “surface” as used herein refers to any generally two-dimensional structure on a solid substrate and may have steps, ridges, kinks, terraces, and the like without ceasing to be a surface.

It should also be understood that, unless clearly indicated to the contrary, in any methods claimed herein that include more than one step or act, the order of the steps or acts of the method is not necessarily limited to the order in which the steps or acts of the method are recited.

### II. Methods of Diagnosing Autism Spectrum Disorder

In general, methods disclosed herein include detecting one or more markers of ASD in a subject from at least one sample collected from said subject. In some embodiments, detecting one or more markers of ASD disclosed herein can be achieved by obtaining a gene expression profile from a sample collected from a subject. As used herein, the term “gene expression profile” refers to a pattern of genes expressed in a sample at the transcription level. Non-limiting examples of methods of measuring gene expression in a sample suitable for use herein include high-density expression array, DNA microarray, polymerase chain reaction (PCR), reverse transcriptase PCR (RT-PCR), real-time quantitative reverse transcription PCR (qRT-PCR), digital droplet PCR (ddPCR), serial analysis of gene expression (SAGE), Spotted cDNA arrays, GeneChip, spotted oligo arrays, bead arrays, RNA Seq, tiling array, northern blotting, hybridization microarray, in situ hybridization, or any combination thereof. In some aspects, a gene expression profile as disclosed herein can be obtained by any known or future method suitable to assess gene expression.

In some embodiments, detecting one or more markers of ASD disclosed herein can be achieved by measuring protein expression for a panel of proteins from a sample collected from a subject. Non-limiting examples of methods of measuring protein expression in a sample suitable for use herein include Western blotting, enzyme-linked immunosorbent assay (ELISA), mass spectrometry, HPLC, flow cytometry, fluorescence-activated cell sorting (FACS), liquid chromatography-mass spectrometry (LC/MS), immunoelectrophoresis, translation complex profile sequencing (TCP-seq), protein microarray, protein chip, capture arrays, reverse phase protein microarray (RPPA), two-dimensional gel electrophoresis or (2D-PAGE), functional protein microarrays, electrospray ionization (ESI), matrix-assisted laser desorption/ionization (MALDI), or a combination thereof. In some aspects, a protein expression profile as disclosed herein can be obtained by any known or future method suitable to assess protein expression.

It is well known in the field that differential gene expression correlates to differential protein expression. (See, e.g., Edfors F et al., Mol Syst Biol. 2016; 12(10):883, the disclosure of which is incorporated herein). As such, the term “markers of ASD” as used herein is understood to refer to gene markers, protein markers, or any combination thereof. Further, methods disclosed herein can also include obtaining a protein expression profile, a gene expression profile, or both for the one or more markers of ASD detailed herein from a subject having or suspected of having ASD using a sample collected from said subject.

### (a) Markers of ASD

The present disclosure provides for a novel panel of proteins as markers for ASD. As used herein, a “panel of proteins” refers to one or more proteins that are predictive of the risk for developing a pathological condition and/or having a pathological condition. In some embodiments, a panel of proteins herein can be predictive of the risk for developing ASD. In some embodiments, a panel of proteins herein can be predictive of having ASD. In some embodiments, a panel of proteins herein can diagnose a subject as having ASD.

In some embodiments, a panel of proteins herein from a subject having or suspected of having ASD may have at least about one protein to at least about 150 proteins, at least about one protein to at least about 100 proteins, at least about one protein to at least about 50 proteins, at least about one protein to at least about 25 proteins, or at least about one protein to at least about 10 proteins differentially expressed compared to a panel of proteins of a subject diagnosed as not having ASD. As used herein, “differentially expressed” refers to a change in expression level (i.e., decreased or increased) compared to the expression level detected in a control sample. In some embodiments, a panel of proteins herein of a subject having or suspected of having ASD may have up to about 150 proteins, up to about 100 proteins, up to about 50 proteins, up to about 25 proteins, or up to about 10 proteins differentially expressed compared to a panel of proteins of a subject diagnosed as not having ASD. In some embodiments, a panel of proteins herein of a subject having or suspected of having ASD may have at least about one protein to at least about 20 proteins, at least about 2 proteins to at least about 15 proteins, or at least about 4 proteins to at least about 10 proteins differentially expressed by about 1-fold to about 10-fold, about 1.5-fold to about 6-fold, or about 2-fold to about 4-fold compared to a panel of proteins of a subject diagnosed as not having ASD.

In some examples, a panel of proteins herein of a subject having or suspected of having ASD may have at least about one protein to at least about nine proteins differentially expressed compared to a panel of proteins of a subject diagnosed as not having ASD. In some examples, a panel of proteins herein of a subject having or suspected of having ASD may have at least about one protein to at least about nine proteins differentially expressed by about 1-fold to about 10-fold, about 1.5-fold to about 6-fold, or about 2-fold to about 4-fold compared to a panel of proteins of a subject diagnosed as not having ASD. In some examples, a panel of proteins herein of a subject having or suspected of having ASD may have at least about one protein to at least about nine proteins with decreased expression compared to a panel of proteins of a subject diagnosed as not having ASD. In some examples, a panel of proteins herein of a subject having or suspected of having ASD may have at least about one protein to at least about nine proteins with about 1-fold to about 10-fold, about 1.5-fold to about 6-fold, or about 2-fold to about 4-fold decreased expression compared to a panel of proteins of a subject diagnosed as not having ASD. In some examples, a panel of proteins herein of a subject having or suspected of having ASD may have at least about one protein to at least about nine proteins with increased expression compared to a panel of proteins of a subject diagnosed as not having ASD. In some examples, a panel of proteins herein of a subject having or suspected of having ASD may have at least about one protein to at least about nine proteins with about 1-fold to about 10-fold, about 1.5-fold to about 6-fold, or about 2-fold to about 4-fold increased expression compared to a panel of proteins of a subject diagnosed as not having ASD.

In some embodiments, a protein differentially expressed in a panel of proteins of a subject having or suspected of having ASD compared to a panel of proteins of a subject diagnosed as not having ASD may be mitogen-activated protein kinase 14 (MAPK14), immunoglobulin D (IgD), dermatopontin (DERM), ephrin type-B receptor 2 (EPHB2), eukaryotic translation initiation factor 4H (elF-4H), receptor tyrosine kinase-like orphan receptor 1 (ROR1), soluble urokinase-type plasminogen activator receptor (suPAR), arylsulfatase B (ARSB), platelet receptor Gl24 (GI24), or any combination thereof.

In some embodiments, a panel of proteins for diagnosing ASD in a subject having or suspected of having ASD for use herein can be identified by analyzing protein abundance data in samples collected from subjects having ASD and subjects not having ASD. In some embodiments, methods of analyzing protein abundance data in samples as described herein can identify one or more proteins having a higher concentration in the samples collected from subjects having ASD compared to the concentration of the one or more proteins in the samples collected from subjects not having ASD. In some embodiments, a protein having a higher concentration in the samples collected from subjects having ASD compared to the samples collected from subjects not having ASD can be said to have “increased expression.” In some examples, a protein identified as having increased expression in the samples collected from subjects having ASD compared to the samples collected from subjects not having ASD can refer to a protein that is only expressed in the samples collected from subjects having ASD. In some examples, a protein identified as having increased expression in the samples collected from subjects having ASD compared to the samples collected from subjects not having ASD may have an about 1-fold to about 10-fold, about 1.5-fold to about 6-fold, or about 2-fold to about 4-fold increase in protein concentration in the samples collected from subjects having ASD compared to the protein concentration in the samples collected from subjects not having ASD.

In some embodiments, a panel of proteins of a subject having or suspected of having ASD as disclosed herein may have increased expression of suPAR, ARSB, GI24, or any combination thereof compared to a panel of proteins of a subject diagnosed as not having ASD. In some embodiments, a panel of proteins of a subject having or suspected of having ASD as disclosed herein may have an about 1-fold to about 10-fold, about 1.5-fold to about 6-fold, or about 2-fold to about 4-fold increase in protein expression of suPAR, ARSB, GI24, or any combination thereof compared to a panel of proteins of a subject diagnosed as not having ASD.

In some embodiments, a protein having a lower concentration in the samples collected from subjects having ASD compared to the samples collected from subjects not having ASD can be said to have “decreased expression.” In some examples, a protein identified as having decreased expression in the samples collected from subjects having ASD compared to the samples collected from subjects not having ASD can refer to a protein that is only expressed in the samples collected from subjects not having ASD. In some examples, a protein identified as having decreased expression in the samples collected from subjects having ASD compared to the samples collected from subjects not having ASD may have an about 1-fold to about 10-fold, about 1.5-fold to about 6-fold, or about 2-fold to about 4-fold decrease in protein concentration in the samples collected from subjects having ASD compared to the protein concentration in the samples collected from subjects not having ASD.

In some embodiments, a panel of proteins of a subject having or suspected of having ASD as disclosed herein may have decreased expression of MAPK14, IgD, DERM, EPHB2, eIF-4H, ROR1, or any combination thereof compared to a panel of proteins of a subject diagnosed as not having ASD. In some embodiments, a panel of proteins of a subject having or suspected of having ASD as disclosed herein may have an about 1-fold to about 10-fold, about 1.5-fold to about 6-fold, or about 2-fold to about 4-fold decrease in protein expression of MAPK14, IgD, DERM, EPHB2, eIF-4H, ROR1, or any combination thereof compared to a panel of proteins of a subject diagnosed as not having ASD.

In some embodiments, a panel of proteins of a subject having or suspected of having ASD as disclosed herein may have increased expression of suPAR, ARSB, GI24, or any combination thereof and decreased expression of MAPK14, IgD, DERM, EPHB2, eIF-4H, ROR1, or any combination thereof compared to a panel of proteins of a subject diagnosed as not having ASD. In some embodiments, a panel of proteins of a subject having or suspected of having ASD as disclosed herein may have an about 1-fold to about 10-fold, about 1.5-fold to about 6-fold, or about 2-fold to about 4-fold increased expression of suPAR, ARSB, GI24, or any combination thereof and an about an about 1-fold to about 10-fold, about 1.5-fold to about 6-fold, or about 2-fold to about 4-fold decreased expression of MAPK14, IgD, DERM, EPHB2, eIF-4H, ROR1, or any combination thereof compared to a panel of proteins of a subject diagnosed as not having ASD.

In some examples, samples used to identify a panel of proteins for use herein can be serum samples collected from human patients having ASD and human patients not having ASD. In some embodiments, protein abundance data collected from the samples herein can be normalized. One of skill in the art will appreciate that there is a multitude of methods that can be used herein to normalize protein abundance data. In some examples, protein abundance data collected from the samples herein can be normalized by taking a log 10 transform and then z-transformation. In some embodiments, outliers can be removed from the normalized protein abundance data set collected from the samples herein. In some examples, any z-transformed values less than about −3 and greater than about 3 can be clipped to about −3 and about 3, respectively to remove any outliers from the normalized protein abundance data disclosed herein. In some embodiments, a panel of proteins for use herein can be identified from a normalized protein abundance data set described herein by a random forest (RF), a t-test, a correlation-based method, or any combination thereof.

In some embodiments, a panel of proteins of a subject having or suspected of having ASD as disclosed herein may have about one to about five core panel proteins with differential expression predictive of ASD. As used herein, a “core panel protein” may be a protein with differential expression in serum samples collected from human patients having ASD compared to human patients not having ASD as identified by a random forest analysis, t-test analysis, and a correlation analysis with ADOS total scores. In some embodiments, a core panel protein as disclosed herein may be IgD, suPAR, MAPK14, EPHB2, DERM, or any combination thereof. In some embodiments, a panel of proteins of a subject having or suspected of having ASD as disclosed herein may have about one to about five core panel proteins and about one to about four other proteins having differential expression in serum samples collected from human patients having ASD compared to human patients not having ASD as identified by either a random forest analysis, t-test analysis, or a correlation analysis with ADOS total scores. In some aspects, proteins having differential expression in serum samples collected from human patients having ASD compared to human patients not having ASD as identified by either a random forest analysis, t-test analysis, or a correlation analysis with ADOS total scores that may comprise a panel of proteins predictive of ASD as disclosed herein may be ARSB, GI24, eIF-4H, ROR1, or any combination thereof.

In some aspects, proteins having differential expression in serum samples collected from human patients having ASD compared to human patients not having ASD as identified by a random forest analysis that may comprise a panel of proteins predictive of ASD as disclosed herein may be MAPK14, IgD, DERM, EPHB2, ALCAM (activated leukocyte cell adhesion molecule), eIF-4H (eukaryotic translation initiation factor 4H), suPAR, SOST (sclerostin), C6 (complement component 6), calcineurin, or any combination thereof.

In some aspects, proteins having differential expression in serum samples collected from human patients having ASD compared to human patients not having ASD as identified by a t-test analysis that may comprise a panel of proteins predictive of ASD as disclosed herein may be DERM, suPAR, calcineurin, MAPK14, EPHB2, RELT (tumor necrosis factor receptor superfamily member 19L), FCN1 (ficolin 1), IgD, PTN (pleiotrophin), C 1QR1 (complement component C1q receptor), or any combination thereof.

In some aspects, proteins having differential expression in serum samples collected from human patients having ASD compared to human patients not having ASD as identified by a correlation analysis with ADOS total scores that may comprise a panel of proteins predictive of ASD as disclosed herein may be DERM, RELT, EPHB2, MAPK14, prolactin receptor, ROR1 (tyrosine-protein kinase transmembrane receptor), GI24 (platelet receptor Gi24, also known as B7-H5 and stress-induced secreted protein-1 (Sisp-1)), suPAR, ARSB (arylsulfatase B), IgD, or any combination thereof.

In some embodiments, a panel of proteins for diagnosing ASD in a subject having or suspected of having ASD can identified by: analyzing protein abundance data in samples collected from subjects having ASD and subjects not having ASD; normalizing protein abundance data to yield a normalized protein abundance data set; subjecting the normalized protein abundance data set to a random forest (RF), a t-test, and a correlation-based method; comparing the top 10 proteins identified from each of the random forest (RF), the t-test, and the correlation-based method to determine overlap; and designating any protein identified in all three tests as a member of a panel of proteins for diagnosing ASD. In some examples, a protein identified in the random forest (RF), the t-test, and the correlation-based method as described herein can be IgD, suPAR, MAPK14, EPHB2, DERM, or any combination thereof.

In some embodiments, a panel of proteins herein of a subject having or suspected of having ASD may have a about 1-fold to about 10-fold, about 1.5-fold to about 6-fold, or about 2-fold to about 4-fold difference in protein expression of MAPK14, IgD, DERM, EPHB2, eIF-4H, ROR1, suPAR, ARSB, GI24 or any combination thereof compared to a panel of proteins of a subject diagnosed as not having ASD. In some embodiments, a panel of proteins herein of a subject having or suspected of having ASD may have a about 1-fold to about 10-fold, about 1.5-fold to about 6-fold, or about 2-fold to about 4-fold decrease in protein expression of MAPK14, IgD, DERM, EPHB2, eIF-4H, ROR1, or any combination thereof compared to a panel of proteins of a subject diagnosed as not having ASD. In some embodiments, a panel of proteins herein of a subject having or suspected of having ASD may have a about 1-fold to about 10-fold, about 1.5-fold to about 6-fold, or about 2-fold to about 4-fold increase in protein expression of suPAR, ARSB, GI24 or any combination thereof compared to a panel of proteins of a subject diagnosed as not having ASD.

In some embodiments, a panel of proteins of a subject having or suspected of having ASD as disclosed herein may have differential expression of at least one protein involved in regulation of multicellular organismal processes compared to a panel of proteins of a subject diagnosed as not having ASD. In some examples, a panel of proteins of a subject having or suspected of having ASD as disclosed herein may have differential expression of at least one protein involved in regulation of multicellular organismal processes compared to a panel of proteins of a subject diagnosed as not having ASD wherein the protein involved in regulation of multicellular organismal processes can be selected from the group of GI24, suPAR, ARSB, MAPK14, EPHB2, ROR1, IgD, and any combination thereof.

In some embodiments, a panel of proteins of a subject having or suspected of having ASD as disclosed herein may have differential expression of at least one protein involved in positive regulation of molecular functions compared to a panel of proteins of a subject diagnosed as not having ASD. In some examples, a panel of proteins of a subject having or suspected of having ASD as disclosed herein may have differential expression of at least one protein involved in positive regulation of molecular functions compared to a panel of proteins of a subject diagnosed as not having ASD wherein the protein involved in regulation of multicellular organismal processes can be selected from the group GI24, suPAR, MAPK14, EPHB2, ROR1, and any combination thereof.

In some embodiments, a panel of proteins of a subject having or suspected of having ASD as disclosed herein may have differential expression of at least one protein involved in colon epithelial cell migration compared to a panel of proteins of a subject diagnosed as not having ASD. In some examples, a panel of proteins of a subject having or suspected of having ASD as disclosed herein may have differential expression of at least one protein involved in colon epithelial cell migration compared to a panel of proteins of a subject diagnosed as not having ASD wherein the protein involved in colon epithelial cell migration is ARSB.

In some embodiments, a panel of proteins of a subject having or suspected of having ASD as disclosed herein may have differential expression of at least one protein involved in negative regulation of CD8-positive, alpha-beta T cell proliferation compared to a panel of proteins of a subject diagnosed as not having ASD. In some examples, a panel of proteins of a subject having or suspected of having ASD as disclosed herein may have differential expression of at least one protein involved in negative regulation of CD8-positive, alpha-beta T cell proliferation compared to a panel of proteins of a subject diagnosed as not having ASD wherein the protein involved in negative regulation of CD8-positive, alpha-beta T cell proliferation is GI24.

In some embodiments, a panel of proteins of a subject having or suspected of having ASD as disclosed herein may have differential expression of at least one protein involved in negative regulation of NMDA glutamate receptor activity compared to a panel of proteins of a subject diagnosed as not having ASD. In some examples, a panel of proteins of a subject having or suspected of having ASD as disclosed herein may have differential expression of at least one protein involved in negative regulation of NMDA glutamate receptor activity compared to a panel of proteins of a subject diagnosed as not having ASD wherein the protein involved in negative regulation of NMDA glutamate receptor activity is EPHB2.

In some embodiments, a panel of proteins of a subject having or suspected of having ASD as disclosed herein may have differential expression of at least one protein involved in neuron projection retraction compared to a panel of proteins of a subject diagnosed as not having ASD. In some examples, a panel of proteins of a subject having or suspected of having ASD as disclosed herein may have differential expression of at least one protein involved in neuron projection retraction compared to a panel of proteins of a subject diagnosed as not having ASD wherein the protein involved in neuron projection retraction is EPHB2.

In some embodiments, a panel of proteins of a subject having or suspected of having ASD as disclosed herein may have differential expression of at least one protein involved in positive regulation of collagen catabolic process compared to a panel of proteins of a subject diagnosed as not having ASD. In some examples, a panel of proteins of a subject having or suspected of having ASD as disclosed herein may have differential expression of at least one protein involved in positive regulation of collagen catabolic process compared to a panel of proteins of a subject diagnosed as not having ASD wherein the protein involved in positive regulation of collagen catabolic process is GI24.

In some embodiments, a panel of proteins of a subject having or suspected of having ASD as disclosed herein may have differential expression of at least one protein involved in positive regulation of multicellular organismal processes compared to a panel of proteins of a subject diagnosed as not having ASD. In some examples, a panel of proteins of a subject having or suspected of having ASD as disclosed herein may have differential expression of at least one protein involved in positive regulation of multicellular organismal processes compared to a panel of proteins of a subject diagnosed as not having ASD wherein the protein involved in positive regulation of multicellular organismal processes can be selected from the group of GI24, ARSB, MAPK14, EPHB2, IgD, and any combination thereof.

In some embodiments, a panel of proteins of a subject having or suspected of having ASD as disclosed herein may have differential expression of at least one protein involved in positive regulation of cell differentiation compared to a panel of proteins of a subject diagnosed as not having ASD. In some examples, a panel of proteins of a subject having or suspected of having ASD as disclosed herein may have differential expression of at least one protein involved in positive regulation of cell differentiation compared to a panel of proteins of a subject diagnosed as not having ASD wherein the protein involved in positive regulation of cell differentiation can be selected from the group of GI24, ARSB, MAPK14, EPHB2, and any combination thereof.

In some embodiments, a panel of proteins of a subject having or suspected of having ASD as disclosed herein may have differential expression of at least one protein involved in locomotion compared to a panel of proteins of a subject diagnosed as not having ASD. In some examples, a panel of proteins of a subject having or suspected of having ASD as disclosed herein may have differential expression of at least one protein involved in locomotion compared to a panel of proteins of a subject diagnosed as not having ASD wherein the protein involved in locomotion can be selected from the group of GI24, suPAR, ARSB, MAPK14, EPHB2, and any combination thereof.

In some embodiments, a panel of proteins of a subject having or suspected of having ASD as disclosed herein may have differential expression of at least one protein involved in the regulation of peptidase activity compared to a panel of proteins of a subject diagnosed as not having ASD. In some examples, a panel of proteins of a subject having or suspected of having ASD as disclosed herein may have differential expression of at least one protein involved in the regulation of peptidase activity compared to a panel of proteins of a subject diagnosed as not having ASD wherein the protein involved in the regulation of peptidase activity can be selected from the group of GI24, suPAR, MAPK14, and any combination thereof.

In some embodiments, a panel of proteins of a subject having or suspected of having ASD as disclosed herein may have differential expression of at least one protein involved in inactivation of MAPKK activity compared to a panel of proteins of a subject diagnosed as not having ASD. In some examples, a panel of proteins of a subject having or suspected of having ASD as disclosed herein may have differential expression of at least one protein involved in inactivation of MAPKK activity compared to a panel of proteins of a subject diagnosed as not having ASD wherein the protein involved in inactivation of MAPKK activity can be EPHB2.

In some embodiments, a panel of proteins of a subject having or suspected of having ASD as disclosed herein may have differential expression of at least one protein involved in intestinal epithelial cell migration compared to a panel of proteins of a subject diagnosed as not having ASD. In some examples, a panel of proteins of a subject having or suspected of having ASD as disclosed herein may have differential expression of at least one protein involved in intestinal epithelial cell migration compared to a panel of proteins of a subject diagnosed as not having ASD wherein the protein involved in intestinal epithelial cell migration can be ARSB.

In some embodiments, a panel of proteins of a subject having or suspected of having ASD as disclosed herein may have differential expression of at least one protein involved in negative regulation of glutamate receptor signaling pathway compared to a panel of proteins of a subject diagnosed as not having ASD. In some examples, a panel of proteins of a subject having or suspected of having ASD as disclosed herein may have differential expression of at least one protein involved in negative regulation of glutamate receptor signaling pathway compared to a panel of proteins of a subject diagnosed as not having ASD wherein the protein involved in negative regulation of glutamate receptor signaling pathway can be EPHB2.

In some embodiments, a panel of proteins of a subject having or suspected of having ASD as disclosed herein may have differential expression of at least one protein involved in the urokinase plasminogen activator signaling pathway compared to a panel of proteins of a subject diagnosed as not having ASD. In some examples, a panel of proteins of a subject having or suspected of having ASD as disclosed herein may have differential expression of at least one protein involved in the urokinase plasminogen activator signaling pathway compared to a panel of proteins of a subject diagnosed as not having ASD wherein the protein involved in the urokinase plasminogen activator signaling pathway can be suPAR.

In some embodiments, a panel of proteins of a subject having or suspected of having ASD as disclosed herein may have differential expression of at least one protein involved in regulation of multicellular organismal development compared to a panel of proteins of a subject diagnosed as not having ASD. In some examples, a panel of proteins of a subject having or suspected of having ASD as disclosed herein may have differential expression of at least one protein involved in regulation of multicellular organismal development compared to a panel of proteins of a subject diagnosed as not having ASD wherein the protein involved in regulation of multicellular organismal development can be selected from the group of GI24, ARSB, MAPK14, EPHB2, ROR1, and any combination thereof.

In some embodiments, a panel of proteins of a subject having or suspected of having ASD as disclosed herein may have differential expression of at least one protein involved in neutrophil degranulation compared to a panel of proteins of a subject diagnosed as not having ASD. In some examples, a panel of proteins of a subject having or suspected of having ASD as disclosed herein may have differential expression of at least one protein involved in neutrophil degranulation compared to a panel of proteins of a subject diagnosed as not having ASD wherein the protein involved in neutrophil degranulation can be selected from the group of suPAR, ARSB, MAPK14, and any combination thereof.

In some embodiments, a panel of proteins of a subject having or suspected of having ASD as disclosed herein may have differential expression of at least one protein involved in immune response compared to a panel of proteins of a subject diagnosed as not having ASD. In some examples, a panel of proteins of a subject having or suspected of having ASD as disclosed herein may have differential expression of at least one protein involved in immune response compared to a panel of proteins of a subject diagnosed as not having ASD wherein the protein involved in neutrophil activation involved in immune response can be selected from the group of suPAR, ARSB, MAPK14, and any combination thereof.

In some embodiments, a panel of proteins of a subject having or suspected of having ASD as disclosed herein may have differential expression of at least one protein involved in neutrophil activation compared to a panel of proteins of a subject diagnosed as not having ASD. In some examples, a panel of proteins of a subject having or suspected of having ASD as disclosed herein may have differential expression of at least one protein involved in neutrophil activation compared to a panel of proteins of a subject diagnosed as not having ASD wherein the protein involved in neutrophil activation can be selected from the group of suPAR, ARSB, MAPK14, and any combination thereof.

In some embodiments, a panel of proteins of a subject having or suspected of having ASD as disclosed herein may have differential expression of at least one protein involved in neutrophil mediated immunity compared to a panel of proteins of a subject diagnosed as not having ASD. In some examples, a panel of proteins of a subject having or suspected of having ASD as disclosed herein may have differential expression of at least one protein involved in neutrophil mediated immunity compared to a panel of proteins of a subject diagnosed as not having ASD wherein the protein involved in neutrophil mediated immunity can be selected from the group of suPAR, ARSB, MAPK14, and any combination thereof.

One of skill in the art will appreciate that protein expression of the panel of protein markers for ASD as disclosed herein can be measured by any suitable means known in the art. In some embodiments, expression of a panel of protein markers for ASD as disclosed herein can be obtained by proteomics. In some examples, expression of a panel of protein markers for ASD as disclosed herein can be obtained by use of an aptamer-based protein array assay (e.g., SOMAscan™).

### (b) Subjects and Samples

In some embodiments, gene and/or protein expression of a panel of protein markers for ASD can be obtained using at least one sample from a subject. As used herein, a suitable subject includes a mammal, a human, a livestock animal, a companion animal, a lab animal, or a zoological animal. In some embodiments, a subject may be a rodent, e.g., a mouse, a rat, a guinea pig, etc. In other embodiments, a subject may be a livestock animal. Non-limiting examples of suitable livestock animals may include pigs, cows, horses, goats, sheep, llamas and alpacas. In yet other embodiments, a subject may be a companion animal. Non-limiting examples of companion animals may include pets such as dogs, cats, rabbits, and birds. In yet other embodiments, a subject may be a zoological animal. As used herein, a “zoological animal” refers to an animal that may be found in a zoo. Such animals may include non-human primates, large cats, wolves, and bears. In other embodiments, the animal is a laboratory animal. Non-limiting examples of a laboratory animal may include rodents, canines, felines, and non-human primates. In some embodiments, the animal is a rodent. Non-limiting examples of rodents may include mice, rats, guinea pigs, etc. In preferred embodiments, a subject of any of the methods disclosed herein may be a mammal (e.g., a human or a non-human primate).

In some embodiments, a subject of any of the methods disclosed herein may be a human patient. In some embodiments, a subject of any of the methods disclosed herein may be a human child patient. Such a child patient is younger than 16 years. In some examples, a child patient subject to methods disclosed herein can have an age younger than 12, for example, younger than 10, 8, 6, 4 or 2. In some examples, the child patient is an infant, e.g., younger than 12 months. Alternatively, the subject may be a human adolescent patient (e.g., 16-20 years old) or a human adult patient. In some embodiments, a subject of any of the methods disclosed herein may be a human male patient. In some embodiments, a subject of any of the methods disclosed herein may be a human male child patient.

In some embodiments, gene and/or protein expression of one or more markers of ASD can be obtained using at least one biological sample from a subject. In some embodiments, at least one biological sample can be obtained from a subject who has not been diagnosed with ASD. In some embodiments, at least one biological sample can be obtained from a subject suspected of having ASD. In some embodiments, at least one biological sample can be obtained from a subject who presents with at least one symptom of ASD. Non-limiting symptoms of ASD may include: making little or inconsistent eye contact; tending not to look at or listen to people; rarely sharing enjoyment of objects or activities by pointing or showing things to others; failing to, or being slow to, respond to someone calling their name or to other verbal attempts to gain attention; having difficulties with the back and forth of conversation; often talking at length about a favorite subject without noticing that others are not interested or without giving others a chance to respond; having facial expressions, movements, and gestures that do not match what is being said; having an unusual tone of voice that may sound sing-song or flat and robot-like; having trouble understanding another person's point of view or being unable to predict or understand other people's actions; repeating certain behaviors or having unusual behaviors (for example, repeating words or phrases, i.e., echolalia); having a lasting intense interest in certain topics, such as numbers, details, or facts; having overly focused interests, such as with moving objects or parts of objects; getting upset by slight changes in a routine; being more or less sensitive than other people to sensory input, such as light, noise, clothing, or temperature; sleep problems; irritability; being able to learn things in detail and remember information for long periods of time; having strong visual and auditory learning capabilities; excelling in math, science, music, or art; or any combination thereof.

In some embodiments, a subject herein can be diagnosed as having ASD by determining one or more non-genetic factors of ASD. As such, methods disclosed herein can further include detecting for one or more non-genetic factors of ASD. In some embodiments, methods herein can test for one or more non-genetic factors of ASD in a subject before obtaining a biological sample to detect for markers of ASD. In some embodiments, methods herein can test for one or more non-genetic factors of ASD in a subject after obtaining a biological sample to detect for markers of ASD. In some embodiments, methods herein can test for one or more non-genetic factors of ASD in a subject while a biological sample is being processed for detection of markers of ASD. One or more non-genetic factors of ASD can be a psychosocial factor, a psychophysical factor, or any combination thereof.

Exemplary psychosocial factors of ASD may include, but are not limited to, anxiety, depression, somatization, stress, cognition, and pain perception. Examples of psychophysical factors can include, but are not limited to, pressure pain threshold (PPT), conditioned pain modulation (CPM), and tactile abnormalities. Any of the psychosocial factors and/or psychophysical factors of a subject can be assessed herein by conventional practice. One or more non-genetic factors of ASD can be determined according to Autism Diagnostic Observation Schedule (ADOS), Autism Diagnostic Interview-Revised (ADI-R), the Adaptive Behavior Assessment System-Second Edition (ABAS-II), and/or Diagnostic and Statistical Manual of Mental Disorders (DSM-5) Autism Diagnostic Criteria.

ADOS is a standardized diagnostic test for Autism Spectrum Disorder (ASD). Trained professionals can administer the ADOS diagnostic screening—a process that involves making direct observations under controlled circumstances that other clinicians are able to replicate. ADOS consists of four different modules designed to provide the most appropriate test for an individual at a certain age or functional level. The modules are: (1) Module One—Designed for individuals who do not have consistent verbal communication skills and uses entirely non-verbal scenarios for scoring; (2) Module Two—Designed for individuals who have minimal verbal communication skills. This may include young children at age-appropriate skill levels; most scenarios require moving around the room and interacting with objects; (3) Module Three—Designed for individuals who are verbally fluent and capable of playing with age-appropriate toys. Can be conducted largely at a desk or table; and (4) Module Four—Designed for individuals who are verbally fluent but beyond the age of playing with toys, incorporating some Module Three elements but also more conversational aspects regarding daily living experiences. Typically, ADOS diagnostic screening is recorded on video so a team can review it and make the diagnosis to help eliminate otherwise subjective biases that are inherent in any individual clinician's work. In some embodiments, at least one biological sample can be obtained from a subject who has been subjected to ADOS, will be subjected to ADOS, or is being subjected to ADOS as part of any one of the methods disclosed herein. In some examples, ADOS diagnostic algorithms can have two behavioral domains: Social Affect (SA) and Restricted and Repetitive Behaviors (RRB) to determine an ADOS total score, which provides a continuous measure of overall ASD symptom severity. In some embodiments, a continuous measure of overall ASD symptom severity can be performed before collecting a biological sample from a subject, after collecting a biological sample from a subject, or as part of any one of the methods disclosed herein.

Autism Diagnostic Interview-Revised (ADI-R), focuses on three functional domains: Language/Communication; Reciprocal Social Interactions; Restricted, Repetitive and Stereotyped Behaviors and Interests. Following highly standardized procedures, an interviewer records and codes the informant's responses to questions covering eight content areas, including: the subject's background, including family, education, previous diagnoses and medications; overview of the subject's behavior; early development and developmental milestones; language acquisition and loss of language or other skills; current functioning in regard to language and communication; social development and play; interests and behaviors; clinically relevant behaviors, such as aggression, self-injury and possible epileptic features. The interview method of ADI-R can be used to assess both children and adults, as long as their mental age is above 2 years, 0 months. In some embodiments, at least one biological sample can be obtained from a subject who has been subjected to ADI-R, will be subjected to ADI-R, or is being subjected to ADI-R as part of any one of the methods disclosed herein.

The Adaptive Behavior Assessment System-Second Edition (ABAS-II) is a multidimensional and standardized assessment tool used to assess the functional skills necessary for daily living of individuals between 0 to 89 years of age. ABAS-II can assess the following skill areas: communication: speech language and communication skills needed for communication with others; community use: skills needed for functioning in the community; functional academics: functional pre-academics and academics; school/home living: skills needed for basic care of a home/living or school/classroom setting; health and safety: skills needed for protection of health and to respond to illness and injury; leisure: skills needed for engaging in and planning leisure and recreational activities; self-care: skills needed for personal care; self-direction: skills needed for independence, responsibility and self-control; social: skills needed to interact socially and get along with other people; motor: basic fine and gross motor skills needed for locomotion, manipulation of the environment and development of more complex activities; and/or skills needed for successful functioning and holding a part- or full-time job in a work setting. The ABAS-II includes five rating forms to be completed by a Parent/Primary Caregiver (ages 0-5), Parent Form (ages 5-21), Teacher/Daycare Provider Form (ages 2-5), Teacher Form (ages 5-21), and Adult Form (ages 16-89). Information obtained from the ABAS-II can be used by psychologists to aid with the diagnosis of disabilities and disorders, identify strengths and weaknesses, and document and monitor an individual's progress over time. In some embodiments, at least one biological sample can be obtained from a subject who has been subjected to ABAS-II, will be subjected to ABAS-II, or is being subjected to ABAS-II as part of any one of the methods disclosed herein.

In some embodiments, at least one biological sample to be used herein can be obtained from a subject having at least one Diagnostic and Statistical Manual of Mental Disorders (DSM-5) Autism Diagnostic Criteria. DSM-5 Autism Diagnostic Criteria include: A) Persistent deficits in social communication and social interaction across multiple contexts, as manifested by the following, currently or by history; B) Restricted, repetitive patterns of behavior, interests, or activities, as manifested by at least two of the following, currently or by history; C) Symptoms must be present in the early developmental period (but may not become fully manifest until social demands exceed limited capacities or may be masked by learned strategies in later life); D) Symptoms cause clinically significant impairment in social, occupational, or other important areas of current functioning; E) These disturbances are not better explained by intellectual disability (intellectual developmental disorder) or global developmental delay. Intellectual disability and autism spectrum disorder frequently co-occur; to make comorbid diagnoses of autism spectrum disorder and intellectual disability, social communication should be below that expected for general developmental level. In some embodiments, at least one biological sample can be obtained from a subject who has demonstrated at least one of the DSM-5 Autism Diagnostic Criteria. In some embodiments, at least one biological sample can be obtained from a subject before the subject has demonstrated at least one of the DSM-5 Autism Diagnostic Criteria, after the subject has demonstrated at least one of the DSM-5 Autism Diagnostic Criteria, or is being assessed for any of the DSM-5 Autism Diagnostic Criteria as part of any one of the methods disclosed herein. In some embodiments, at least one biological sample to be used herein can be obtained from a subject having a level 1, 2, or 3 severity level for ASD according to the DSM-5. A description of grading ASD according to the DSM-5 is provided in Table 1. In some embodiments, at least one biological sample can be obtained from a subject before the subject has demonstrated a level 1, 2, or 3 severity level for ASD, after the subject has demonstrated a level 1, 2, or 3 severity level for ASD, or is being assessed for level 1, 2, or 3 severity level for ASD as part of any one of the methods disclosed herein.

TABLE 1
Severity levels for autism spectrum disorder

| Severity Level | Social communication | Restricted, repetitive behaviors |
|---|---|---|
| Level 3 | Severe deficits in verbal and | Inflexibility of behavior, extreme |
| “Requiring very | nonverbal social communication | difficulty coping with change, or |
| substantial support” | skills cause severe impairments in | other restricted/repetitive |
| | functioning, very limited initiation | behaviors markedly interfere |
| | of social interactions, and minimal | with functioning in all spheres. |
| | response to social overtures from | Great distress/difficulty changing |
| | others. For example, a person with | focus or action. |
| | few words of intelligible speech | |
| | who rarely initiates interaction | |
| | and, when he or she does, makes | |
| | unusual approaches to meet needs | |
| | only and responds to only very | |
| | direct social approaches | |
| Level 2 | Marked deficits in verbal and | Inflexibility of behavior, |
| “Requiring | nonverbal social communication | difficulty coping with change, or |
| substantial support” | skills; social impairments apparent | other restricted/repetitive |
| | even with supports in place; | behaviors appear frequently |
| | limited initiation of social | enough to be obvious to the |
| | interactions; and reduced or | casual observer and interfere with |
| | abnormal responses to social | functioning in a variety of |
| | overtures from others. For | contexts. Distress and/or difficult |
| | example, a person who speaks | changing focus or action. |
| | simple sentences, whose | |
| | interaction is limited to narrow | |
| | special interests, and how has | |
| | markedly odd nonverbal | |
| | communication. | |
| Level 1 | Without supports in place, deficits | Inflexibility of behavior causes |
| “Requiring support” | in social communication cause | significant interference with |
| | noticeable impartments. Difficulty | functioning in one or more |
| | initiating social interactions, and | contexts. Difficulty switching |
| | clear examples of atypical or | between activities. Problems of |
| | unsuccessful response to social | organization and planning hamper |
| | overtures of others. May appear to | independence. |
| | have decreased interest in social | |
| | interactions. For example, a person | |
| | who is able to speak in full | |
| | sentences and engages in | |
| | communication but whose to-and- | |
| | fro conversation with others fails, | |
| | and whose attempts to make | |
| | friends are odd and typically | |
| | unsuccessful. | |

In some embodiments, a biological sample obtained from a subject to obtain a gene expression profile and/or to determine protein expression as disclosed herein may be a tissue sample, a blood sample, a plasma sample, a hair sample, venous tissues, cartilage, a sperm sample, a skin sample, an amniotic fluid sample, a buccal sample, saliva, urine, serum, sputum, bone marrow or any combination thereof. In some examples, a biological sample obtained from a subject to obtain a gene expression profile and/or to determine protein expression as disclosed herein can be a whole blood sample, a blood serum sample, a blood plasma sample, or any combination thereof.

In some aspects, a biological sample obtained from a subject to obtain a gene expression profile and/or a protein expression profile as disclosed herein may be stored at about 25° C. to about −80° C. for up to about 1 day to about 2 years, about 1 week to about 1 year, or about 1 month to about 6 months. In other aspects, a biological sample obtained from a subject may be immediately processed to obtain a gene expression profile and/or a protein expression profile as disclosed herein. Non-limiting examples of biological sample preparation methods can be found in art, for example in Gallagher & Wiley, (2012). CURRENT PROTOCOLS ESSENTIAL LABORATORY TECHNIQUES. Hoboken, N.J.: Wiley-Blackwell, the disclosure of which is incorporated herein in its entirety.

In some aspects, a gene expression profile and/or protein expression as disclosed herein can be obtained using a biological sample from a subject wherein the data is compared to a control biological sample. In some aspects, a control biological sample may be from a subject that has not been diagnosed with ASD. In some aspects, a gene expression profile obtained from a control biological sample can be compared to a gene expression profile obtained from a subject diagnosed with or suspected of having ASD to analyze for differential gene expression. In some aspects, protein expression of one or more proteins obtained from a control biological sample can be compared to the protein expression of the one or more proteins obtained from a subject diagnosed with or suspected of having ASD to analyze for differential protein expression. As used herein, the term “differential expression analysis” refers to a method of taking the normalized read count data and performing statistical analysis to discover quantitative changes in expression levels between a subject with ASD or suspected of having ASD and a control subject. Selection of a method of differential expression analysis suitable for use herein can depend on, but is not limited to, the method of used to obtain the data, experimental design, number of data sets to be compared, or any combination thereof.

III. Methods of Diagnosing and/or Treating ASD in a Subject

In some aspects, provided herein are methods for diagnosing ASD in a subject based on any of the markers of ASD disclosed herein, non-genetic factors, or any combination thereof. Such methods may comprise determining the levels of at least one marker of ASD, wherein the at least one marker of ASD can be selected from MAPK14, IgD, DERM, EPHB2, eIF-4H, ROR1, suPAR, ARSB, GI24, or any combination thereof Alternatively, methods for diagnosing ASD may further include analyzing a subject's non-genetic profile as disclosed herein by assessing Autism Diagnostic Observation Schedule (ADOS), Autism Diagnostic Interview-Revised (ADI-R), the Adaptive Behavior Assessment System-Second Edition (ABAS-II), and/or Diagnostic and Statistical Manual of Mental Disorders (DSM-5) Autism Diagnostic Criteria. Based on the diagnosis as assessed by any of the methods disclosed herein, a tailored treatment approach can be developed and administered to that subject.

In some embodiments, a subject having or suspected of having ASD having a panel of proteins as assessed by the methods herein with increased expression of suPAR, ARSB, GI24, or any combination thereof as compared to a panel of proteins of a subject diagnosed as not having ASD can be diagnosed as having ASD. In some embodiments, a subject having or suspected of having ASD having a panel of proteins as assessed by the methods herein with decreased expression of MAPK14, IgD, DERM, EPHB2, eIF-4H, ROR1, or any combination thereof as compared to a panel of proteins of a subject diagnosed as not having ASD can be diagnosed as having ASD. In some embodiments, a subject having or suspected of having ASD having a panel of proteins as assessed by the methods herein with decreased expression of at least one, at least two, at least three, or at least four proteins selected from IgD, MAPK14, EPHB2, and DERM as compared to a panel of proteins of a subject diagnosed as not having ASD can be diagnosed as having ASD. In some embodiments, a subject having or suspected of having ASD having a panel of proteins as assessed by the methods herein with decreased expression of at least one, at least two, at least three, or at least four proteins selected from IgD, MAPK14, EPHB2, and DERM and increased expression of suPAR as compared to a panel of proteins of a subject diagnosed as not having ASD can be diagnosed as having ASD.

In some embodiments, a subject having or suspected of having ASD having a panel of proteins as assessed by the methods herein with decreased expression of at least one protein selected from IgD, MAPK14, EPHB2, and DERM and decreased expression of at least one protein selected from eIF-4H and ROR1 as compared to a panel of proteins of a subject diagnosed as not having ASD can be diagnosed as having ASD. In some embodiments, a subject having or suspected of having ASD having a panel of proteins as assessed by the methods herein with decreased expression of at least two proteins selected from IgD, MAPK14, EPHB2, and DERM decreased expression of at least one protein selected from eIF-4H and ROR1 and increased expression of at least one protein selected from suPAR, ARSB and GI24 as compared to a panel of proteins of a subject diagnosed as not having ASD can be diagnosed as having ASD. In some embodiments, a subject having or suspected of having ASD having a panel of proteins as assessed by the methods herein with decreased expression of at least three proteins selected from IgD, MAPK14, EPHB2, and DERM, decreased expression of at least one protein selected from eIF-4H and ROR1 and increased expression of at least one protein selected from suPAR, ARSB and GI24 as compared to a panel of proteins of a subject diagnosed as not having ASD can be diagnosed as having ASD. In some embodiments, a subject having or suspected of having ASD having a panel of proteins as assessed by the methods herein with decreased expression of at least three proteins selected from IgD, MAPK14, EPHB2, and DERM, decreased expression of at least one protein selected from eIF-4H and ROR1 and increased expression of at least one protein selected from suPAR, ARSB and GI24 as compared to a panel of proteins of a subject diagnosed as not having ASD can be diagnosed as having ASD. In some embodiments, a subject having or suspected of having ASD having a panel of proteins as assessed by the methods herein with decreased expression of IgD, MAPK14, EPHB2, and DERM, decreased expression of at least one protein selected from eIF-4H and ROR1, and increased expression of at least one protein selected from suPAR, ARSB and GI24 as compared to a panel of proteins of a subject diagnosed as not having ASD can be diagnosed as having ASD. In some embodiments, a subject having or suspected of having ASD having a panel of proteins as assessed by the methods herein with decreased expression of IgD, MAPK14, EPHB2, and DERM, decreased expression of at least one protein selected from eIF-4H and ROR1, increased expression of suPAR, and increased expression of at least one protein selected from ARSB and GI24 as compared to a panel of proteins of a subject diagnosed as not having ASD can be diagnosed as having ASD. In some embodiments, a subject having or suspected of having ASD having a panel of proteins as assessed by the methods herein with decreased expression of IgD, MAPK14, EPHB2, DERM, eIF-4H and ROR1 and increased expression of suPAR, ARSB and GI24 as compared to a panel of proteins of a subject diagnosed as not having ASD can be diagnosed as having ASD.

In some embodiments, area under the curve (AUC) can be used to evaluate the diagnostic accuracy. In some embodiments, area under the curve (AUC) can be used to quantify the overall ability of a test to discriminate between different sample properties. In some examples herein, AUC can be used to discriminate between subjects with ASD and those without ASD.

In some embodiments, a decrease of at least 9 proteins and an increase of at least one protein of the RF-based panel of proteins (e.g., MAPK14, IgD, DERM, EPHB2, ALCAM, eIF-4H, SOST, C6, calcineurin, and suPAR) in a sample collected from a subject herein can discriminate between not having ASD and having ASD with an AUC of about 0.839. In some embodiments, a decrease of MAPK14, IgD, DERM, EPHB2, ALCAM, eIF-4H, SOST, C6 and calcineurin and an increase of suPAR in a sample collected from a subject herein can discriminate between not having ASD and having ASD with about 70% to about 90% probability (e.g., about 70%, about 75%, about 80%, about 85%, about 90%). In some examples, a decrease of MAPK14, IgD, DERM, EPHB2, ALCAM, eIF-4H, SOST, C6 and calcineurin and an increase of suPAR in a sample collected from a subject herein can discriminate between not having ASD and having ASD with about 84% probability.

In some embodiments, a decrease of at least 8 proteins and an increase of at least two proteins of the t-test-based panel of proteins (e.g., of DERM, calcineurin, MAPK14, EPHB2, RELT, IgD, PTN, C1QR1, FCN1, and suPAR) in a sample collected from a subject herein can discriminate between not having ASD and having ASD with an AUC of about 0.837. In some embodiments, a decrease of DERM, calcineurin, MAPK14, EPHB2, RELT, FCN1, IgD, PTN, and C1QR1 and an increase of FCN1 and/or suPAR in a sample collected from a subject herein can discriminate between not having ASD and having ASD with about 70% to about 90% probability (e.g., about 70%, about 75%, about 80%, about 85%, about 90%). In some examples, a decrease of MAPK14, IgD, DERM, EPHB2, ALCAM, eIF-4H, SOST, C6 and calcineurin and an increase of and/or suPAR in a sample collected from a subject herein can discriminate between not having ASD and having ASD with about 84% probability.

In some embodiments, a decrease of at least 7 proteins and an increase of at least three proteins of the correlation-based panel of proteins (e.g., DERM, RELT, EPHB2, MAPK14, prolactin receptor, ROR1, IgD, ARSB, GI24, and suPAR) in a sample collected from a subject herein can discriminate between not having ASD and having ASD with an AUC of about 0.853. In some embodiments, a decrease of DERM, RELT, EPHB2, MAPK14, prolactin receptor, ROR1, and IgD and an increase of ARSB, GI24, and/or suPAR in a sample collected from a subject herein can discriminate between not having ASD and having ASD with about 70% to about 90% probability (e.g., about 70%, about 75%, about 80%, about 85%, or about 90%). In some examples, a decrease of DERM, RELT, EPHB2, MAPK14, prolactin receptor, ROR1, and IgD and an increase of ARSB, GI24, and/or suPAR in a sample collected from a subject herein can discriminate between not having ASD and having ASD with about 85% probability.

In some embodiments, differential expression of IgD, MAPK14, EPHB2, DERM, suPAR, ROR1, ARSB, GI24, and eIF-4H in a sample collected from a subject herein can discriminate between not having ASD and having ASD with an AUC of about 0.86. In some embodiments, differential expression of IgD, MAPK14, EPHB2, DERM, suPAR, ROR1, ARSB, GI24, and eIF-4H in a sample collected from a subject herein can discriminate between not having ASD and having ASD with about 80% to about 99% probability (e.g., about 80%, about 85%, about 90%, about 95%, or about 99%). In some examples, differential expression of IgD, MAPK14, EPHB2, DERM, suPAR, ROR1, ARSB, GI24, and eIF-4H in a sample collected from a subject herein can discriminate between not having ASD and having ASD with about 86% probability.

A tailored treatment approach for ASD for use herein may include one or more of the following: applied behavior analysis; cognitive behavior therapy; early intervention; educational and school-based therapies; joint attention therapy; pharmaceutical treatment; dietary therapy; occupational therapy; parent-mediated therapy; physical therapy; social skills training; and speech therapy.

Applied behavior analysis for ASD tries to reinforce wanted behaviors and reduce unwanted behaviors. Non-limiting types of behavior management therapy suitable for use herein can be Positive Behavioral and Support (PBS); Pivotal Response Training (PRT); Early Intensive Behavioral Intervention (EMI); Discrete Trial Teaching (DTT); or any combination thereof.

Cognitive behavior therapy focuses on the connection between thoughts, feelings, and behaviors. Cognitive behavior therapy as used herein can be individualized to a subject's strengths and/or weaknesses and can help the deal with anxiety, cope with social situations, and better recognize emotions.

Early intervention therapies as used herein can occur at or before preschool age, as early as 2 or 3 years of age. Early intervention programs often include, but are not limited to: Family training, Speech therapy, Hearing impairment services, Physical therapy, Nutrition services, or any combination thereof.

Educational and school-based therapies as used herein refers to creating a teaching environment designed to meet a subject's specific needs and skills and/or minimize restrictions on the subject's access to typical learning experiences and interactions. In some examples, educating subjects with ASD can often include, but is not limited to, any combination of one-on-one, small group, and regular classroom instruction.

Joint attention therapy as used herein can focus on improving specific skills related to shared attention such as, but not limited to pointing, showing, and coordinating looks between a person and an object.

In some examples, dietary therapy can involve administration of a gluten-free, casein-free, or any combination of gluten-free and casein-free diets.

Occupational therapy can help a subject diagnosed with ASD do everyday tasks by finding ways to work within and make the most of their needs, abilities, and interests such as, but not limited to use of a specially designed computer mouse and keyboard to ease communication or teaching personal care skills such as getting dressed and eating.

In parent-mediated therapy as used herein, parents learn therapy techniques (e.g., joint attention therapy; social communication therapy; behavioral therapy) from professionals and provide specific therapies to their own child diagnosed with ASD.

Physical therapy as used herein can include activities and/or exercises that build motor skills and improve strength, posture, and balance in a subject diagnosed as having ASD.

Social skills training teaches children the skills they need to interact with others. Social skills training as used herein can include repeating and/or reinforcing certain desired behaviors to improve social skills including, but not limited to conversation, handling teasing, being a good sport, showing good host behavior during play dates, or any combination thereof.

Speech-language therapy can help a subject diagnosed as having ASD improve their abilities to communicate and interact with others. In some examples, speech-language therapy as used herein can be verbal skill therapy, nonverbal communication therapy, or any combination thereof. Verbal skill therapy can help a subject diagnosed as having ASD improve, for example, correctly naming people and things, better explaining feelings and emotions, using words and sentences better, and/or improving the rate and rhythm of speech. In some examples, nonverbal communication skill therapy can teach a subject diagnosed as having ASD nonverbal communication skills, such as (but not limited to) using hand signals or sign language and/or using picture symbols to communicate (i.e., Picture Exchange Communication System).

There are no presently known medications (e.g., pharmaceuticals) that can cure autism spectrum disorder (ASD) or all of its symptoms; however, some medications can treat one or more symptoms associated with ASD. In some embodiments, a tailored treatment approach for ASD for use herein may include pharmaceutical therapy. In some examples, a pharmaceutical therapy suitable for use herein can include, but is not limited to, administration of one or more of the following: selective serotonin re-uptake inhibitors (SSRIs) (e.g., citalopram, escitalopram, fluoxetine, paroxetine, sertraline, fluvoxamine, amitriptyline);tricyclics (e.g., amitriptyline, desipramine, imipramine, nortriptyline); psychoactive or anti-psychotic medications (e.g., haloperidol, loxapine, thioridazone, molindone, thiothixene, fluphenazine, mesoridazone, trifluperazine, chlorpromazine, aripiprazole, clozapine, risperidone, quetiapine, olanzapine); stimulants (e.g., amphetamine and dextroamphetamine, pemoline, methylphenidate); anti-anxiety medications (e.g., lorazepam, buspirone, prazepam, propranolol, clonazepam, oxazepam); anticonvulsants (e.g., gab apentin, pregabalin, carbamazepine, lamotrigine, topiramate, felbamate, tiagabine, diazepam rectal, phenobarbital, phenytoin, primidone, valproate, vigabatrin, oxcarbazepine, zonisamide, levetiracetam); steroids (e.g., corticosteroid, prednisolone, betamethasone. dexamethasone, hydrocortisone, methylprednisolone, deflazacort.); or any combination thereof.

A suitable tailored treatment approach for ASD as used herein may be selected based on the subject's diagnosis. In some embodiments, a subject can be diagnosed with ASD based on increased protein expression of one or more markers for ASD according to methods herein. In some embodiments, a subject can be diagnosed with ASD based on increased protein expression of one or more markers for ASD according to methods herein wherein those methods can further include an assessment of at least one non-genetic factor of ASD. In some embodiments, methods of diagnosing ASD using protein markers of ASD in combination with an assessment of at least one non-genetic factor of ASD can diagnosis the severity of ASD in a subject. In some examples, methods herein may diagnose a subject herein as having mild (level 1), moderate (level 2) or severe (level 3) ASD. As such, embodiments herein can provide a suitable tailored treatment approach for ASD selected based on the severity of a subject's ASD diagnosis. For example, if a subject is assessed as having mild (level 1) ASD, one or more psychosocial therapies may be used, either taken alone or in combination with a mild and/or low dose pharmaceutical therapy. On the other hand, if a subject is assessed as having severe (level 3) ASD, tone or more psychosocial therapies may be used, either taken alone or in combination with a strong and/or high dose pharmaceutical therapy. Choosing suitable tailored treatment approaches for subjects having different levels of ASD would be within the knowledge of medical practitioners.

### IV. Kits

The present disclosure provides kits for performing any of the methods disclosed herein. In some aspects, the present disclosure provides a kit for determining expression of one or more markers of ASD as disclosed herein and for diagnosing ASD. Such a kit may comprise a means for determining any of the combinations of ASD markers as disclosed herein.

In some embodiments, the means for determining expression of one or more markers of ASD disclosed herein may have a set of antibodies, peptides, aptamers, or any combination thereof. In some examples, a means for determining expression of one or more markers of ASD disclosed herein may have a set of aptamers. Each of the aptamers can be designed for detecting a target ASD marker in the combination and the whole set, collectively, is designed for detecting all ASD markers (e.g., MAPK14, IgD, DERM, EPHB2, eIF-4H, ROR1, suPAR, ARSB, GI24) in the combination. Design of such an aptamer for detecting a particular protein is within the knowledge of a skilled person in the art. See, e.g., Sambrook et al. et al., MOLECULAR CLONING—A LABORATORY MANUAL (2ND ED.), Vols. 1-3, Cold Spring Harbor Laboratory, Cold Spring Harbor, N.Y. (1989).

In some embodiments, kits disclosed herein can have a solid support member, on which the set of aptamers (e.g., “probes”) can be immobilized. In some examples, kits disclosed herein may comprise a microarray chip comprising a support member, on which the set of aptamers (probes) can be immobilized. The probes may have oligonucleotide or peptide molecules that bind to a specific target molecule. The support member in the microarray chip may be either porous or non-porous. For example, the probes may be attached to a nitrocellulose or nylon membrane or filter covalently at either the 3′ or the 5′ end of the aptamer. Alternatively, the support member may have a glass or plastic surface. In some examples, the solid phase may be a nonporous or, optionally, a porous material such as a gel.

In some embodiments, a microarray chip may comprise a support member with an ordered array of binding (e.g., hybridization) sites or “probes” each representing one of the target protein marker described herein. Preferably the microarrays are addressable arrays, and more preferably positionally addressable arrays. For example, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

The microarray chips disclosed herein can be made in a number of ways. In some examples, the microarray chips are reproducible, allowing multiple copies of a given array to be produced and easily compared with each other. In some examples, microarrays are made from materials that are stable under binding (e.g., hybridization) conditions. The microarrays may be small, e.g., between 1 cm2 and 25 cm2, between 12 cm2 and 13 cm2, or about 3 cm2. However, larger arrays are also contemplated.

Any of the kits disclosed herein may further comprise a container for placing a biological sample, and optionally a tool for collecting a biological sample from a subject. Alternatively or in addition, the kit may further comprise one or more reagents for determining protein levels of the combination of markers of ASD from the biological sample. In some examples, the kit may comprise reagents for immunodetection of ASD markers. In other examples, the kit may comprise reagents for hybridization.

Any of the kits may further comprise an instruction manual providing guidance for using the kit to determine a protein panel having any combination of the target ASD protein markers as disclosed herein.

In some embodiments, the kit may further comprise questionnaires for assessing one or more of the non-genetic factors associated with ASD, e.g., Autism Diagnostic Observation Schedule (ADOS), Autism Diagnostic Interview-Revised (ADI-R), the Adaptive Behavior Assessment System-Second Edition (ABAS-II), and/or Diagnostic and Statistical Manual of Mental Disorders (DSM-5) Autism Diagnostic Criteria. Instructions of how to use such questionnaires for assessing the non-genetic factors may also be included.

Further, any of the kits disclosed herein may comprise a processor, e.g., a computational processor, for ASD assessment. Such a processor may be configured with a regression model such as those disclosed herein. By inputting the marker profile (e.g., the protein expression level of ASD protein markers) and optionally any of the non-genetic factors, the processor may process the information to diagnose ASD and optionally diagnose the level of ASD severity.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1

In this example, a proteomic analysis of serum collected from subjects diagnosed as having and not having Autism spectrum disorder (ASD) using the SomaLogic SOMAScan™ platform, was performed, incorporating machine learning of the associated demographic and clinical data, for biomarker discovery in accordance with in accordance with aspects of the present disclosure. In brief, serum samples were subjected to SomaLogic SOMAScan™ platform using a methodology similar to that disclosed in Shubin et al., Sci Data 6, 314 (2019), the disclosure of which is incorporated herein in its entirety.

For these examples, a total of 154 male pediatric subjects were enrolled. Written informed consent was obtained from all subjects and/or their legal guardians prior to study participation. Subjects with a genetic, metabolic, or other concurrent physical, mental, or neurological disorder were excluded from enrollment. The “ASD group” was comprised of 76 subjects with a mean age of 5.6 years (SD 1.7 years). The “typically developing (TD) group” was comprised of 78 subjects with a mean age of 5.7 years (SD 2.0 years). The ethnic breakdown was as follows: 73 White/Caucasian, 32 Hispanic/Latino, 17 African American/Black, 5 Asian or Pacific Islander, 23 multiple ethnicities or other and 4 not reported (Table 2). Co-morbid/clinical conditions and the use of psychiatric medications are summarized in Table 2.

TABLE 2
Demographic data, co-morbid conditions, and use of psychiatric
medications in ADS and TD subjects.

| | ASD | TD |
|---|---|---|
| | (n = 76) | (n = 78) |
| Age: mean (SD) years | 5.6 (1.7) | 5.7 (2.0) |
| Ethnicity | | |
| White/Caucasian | 33 (45.2%) | 40 (51.9%) |
| Hispanic/Latino | 26 (35.6%) | 6 (7.8%) |
| African American/Black | 3 (4.1%) | 14 (18.2%) |
| Asian or Pacific Islander | 2 (2.6%) | 3 (3.9%) |
| Multiple ethnicities or Other | 9 (12.3%) | 14 (18.2%) |
| Not reported | 3 (4.1%) | 1 (1.2%) |
| Co-morbid conditions* | | |
| None | 38 (52.8%) | 58 (75.3%) |
| ADHD | 2 (2.8%) | 1 (1.3%) |
| Allergies | 30 (41.7%) | 17 (22.4%) |
| Asthma | 2 (2.8%) | 0 (0%) |
| Celiac Disease | 1 (1.4%) | 0 (0%) |
| GERD | 1 (1.4%) | 0 (0%) |
| PTSD | 0 (0%) | 1 (1.3%) |
| Sleep Apnea | 2 (2.8%) | 0 (0%) |
| Not reported | $ (5.6%) | 1 (1.3%) |
| Psychiatric medications | | |
| None | 69 (92%) | 75 (97.4%) |
| Anti-depressant | 2 (2.7%) | 0 (0%) |
| Ant9-psychotic | 0 (0%) | 1 (1.3%) |
| Sedative | 1 (1.3%) | 0 (0%) |
| SSRI | 2 (2.7%) | 0 (0%) |
| Stimulant | 1 (1.3%) | 1 (1.3%) |
| Not reported | 1 (1.3%) | 1 (1.3%) |

*Some subjects reported multiple co-morbid conditions

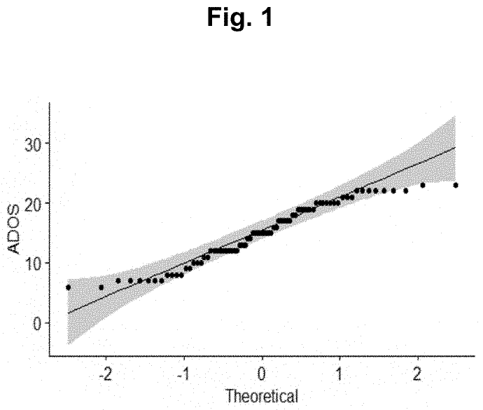

For the ASD group, all subjects were assessed by a clinical psychologist with research-reliability training using both the Autism Diagnostic Observation Schedule (ADOS) and the Autism Diagnostic Interview-Revised (ADI-R). Clinical diagnosis was made based on these data and overall clinical impression using DSM-5 criteria. In addition, ADOS diagnostic algorithms consisting of two behavioral domains: Social Affect (SA) and Restricted and Repetitive Behaviors (RRB) were used to determine an ADOS total score, which provides a continuous measure of overall ASD symptom severity. These scores can be used to compare ASD symptom severity across individuals of different developmental levels and were used in the correlation analyses (FIG. 1). For the TD group, all subjects underwent a developmental screening using the Adaptive Behavior Assessment System-Second Edition (ABAS-II) to rule out developmental concerns. TD subjects were excluded if they had any first- or second-degree relatives diagnosed with ASD.

All subjects were healthy—defined as being fever-free for 24 hours and presenting with no clinical symptoms. A fasting blood draw was performed on ASD and TD subjects between the hours of 8-10 AM in a 3.5 ml Serum Separation Tube using standard venipuncture technique. The blood was gently mixed by 5 inversions and then stored upright for clotting at room temperature for 10-15 minutes. Blood was centrifuged immediately after the clotting time in a swing bucket rotor for 15 minutes at 1,100-1,300 g. After centrifugation was completed and the turbidity and hemolysis of the serum had been recorded, 250 μl aliquots of serum were transferred to 1.0 ml coded cryovials and then stored at −80° C. Serum was shipped on dry ice to location for analysis.

The SOMAScan™ platform 1.3k was used for analysis. SOMAmer aptamer reagents consisted of short single-stranded DNA sequences with ‘protein-like’ appendages that allow tight and specific binding to protein targets. The assay measured 1,317 proteins in 150 μl serum in 154 samples to identify an optimal subset of proteins to be used as a panel for ASD prediction. An additional 14 samples (7 ASD and 7 TD) were included as blinded duplicates to assess the variability of SOMAScan™ analytes. In this study, 192 proteins failed to pass quality control (QC). After removing these proteins, 1,125 proteins were analyzed. The protein abundance data were normalized by taking log 10 transform and then z-transformation. To deal with outliers, any z-transformed values less than −3 and greater than 3 were clipped to −3 and 3, respectively. To discover proteins for ASD prediction, three different methods were deployed: random forest (RF), t-test, and correlation-based methods, as described below.

Random forest (RF) Test. RF, a well-known decision tree-based ensemble learning method, produces consistent results even without hyper-parameter tuning. At the same time, it measures feature importance by observing how random re-shuffling of each predictor influences its model performance. To train RF models and calculate feature importance, an R package, ‘randomForest’ , was used. In this study, the MeanDecreaseGini (mean decrease in Gini Index), a weighted measure of the average reduction in node impurity within a random forest, was chosen as the surrogate representing a protein's importance in predicting ASD versus TD. With the normalized data, an RF model was trained 1,000 times. Each protein's importance value was then averaged over the 1,000 runs. The 10 proteins with the highest averaged importance values were chosen for the RF-based prediction model.

T-Test. A t-test, which determines if there is a significant difference between the means of two groups, is a widely used approach to discover biomarkers in biological data. In this study, the 10 proteins with the most highly significant t-test values were selected for the prediction model.

Correlation-based Methods. A correlation approach, which measures the statistical relationship between two variables, was used to calculate each protein's correlation with Autism Diagnostic Observation Schedule (ADOS) total scores (Social Affect (SA)+Restricted and Repetitive Behaviors (RRB)), as a measure of ASD severity. Based upon the absolute values of each protein's correlation coefficient, the 10 most highly correlated proteins were selected as the correlation-based predictive proteins.

After identifying the top-10 predictive proteins from each of the 3 methods (RF, t-tests and correlation), five were common to all three prediction models used: mitogen-activated protein kinase 14 (MAPK14), immunoglobulin D (IgD), dermatopontin (DERM), ephrin type-B receptor 2 (EPHB2), and soluble urokinase-type plasminogen activator receptor (suPAR) (Table 3).

TABLE 3
Top-10 predictive proteins identified by three different methods
where the 5 core proteins common to all three methods are in bold

| | Random Forest | | T-test | | | Correlation with ADOS total | | |
|---|---|---|---|---|---|---|---|---|
| | | Importance | | log2 fold | | scores | | |
| No. | Protein | value | Protein | change | p-value | Protein | Coefficient | p-value |
| 1 | MAPK14 | 1.4489 | DERM | −0.1505 | 1.3837e−08 | DERM | −0.4131 | 1.0095e−07 |
| 2 | IgD | 1.3883 | suPAR | 0.1000 | 2.5238e−07 | RELT | −0.3852 | 8.0761e−07 |
| 3 | DERM | 1.2726 | Calcineurin | −0.1274 | 3.1577e−07 | EPHB2 | −0.3834 | 9.1825e−07 |
| 4 | EPHB2 | 1.0284 | MAPK14 | −0.0916 | 1.0691e−06 | MAPK14 | −0.3778 | 1.3583e−06 |
| 5 | ALCAM | 0.8565 | EPHB2 | −0.0788 | 8.8167e−07 | Prolactin | 0.3754 | 1.6102e−06 |
| | | | | | | Receptor | | |
| 6 | eIF-4H | 0.8077 | RELT | −0.1123 | 1.0065e−06 | ROR1 | −0.3560 | 5.8703e−06 |
| 7 | suPAR | 0.6558 | FCN1 | 0.1056 | 1.6464e−06 | GI24 | 0.3554 | 6.1107e−06 |
| 8 | SOST | 0.6543 | IgD | −0.8843 | 1.6952e−06 | suPAR | 0.3545 | 6.4619e−06 |
| 9 | C6 | 0.6403 | PTN | −0.0855 | 2.6899e−06 | ARSB | 0.3535 | 6.8678e−06 |
| 10 | Calcineurin | 0.6015 | C1QR1 | −0.1245 | 7.6353e−06 | IgD | −0.3535 | 6.8722e−06 |

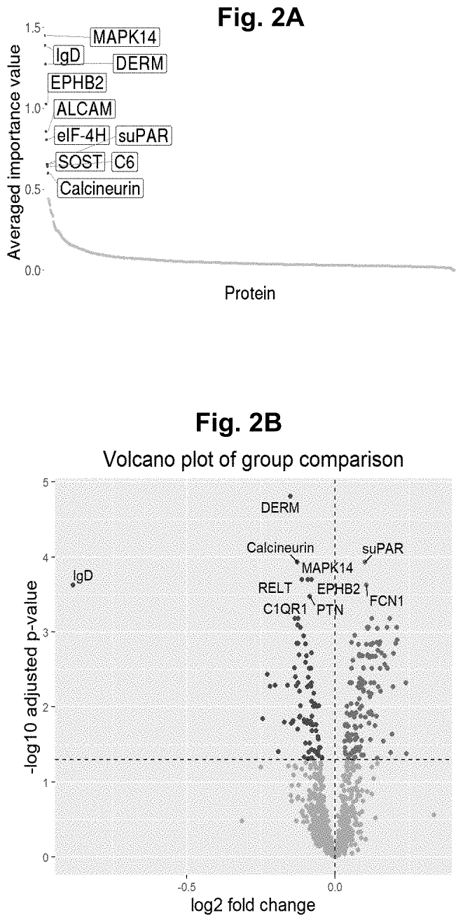

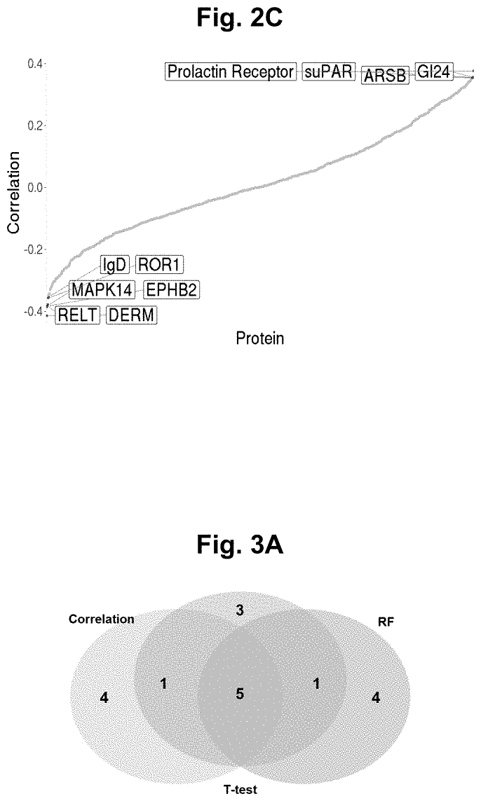

These were considered ‘core’ proteins, leaving 13 additional proteins that were not part of the core. A prediction model trained with the 5 core proteins was taken as a baseline model. FIGS. 2A-2C show the importance plots for the top-10 proteins identified by RF analysis (FIG. 2A), top-10 significant proteins on a volcano plot identified by t-tests (FIG. 2B), and correlation with ADOS total scores (FIG. 2C). The significant correlations between the top-10 highly correlated proteins and the ADOS total scores ranged between −0.4131 and 0.3754.

Next, it was investigated whether the addition of one or more of the 13 proteins provided any additive predictive power. A logistic regression model was used with datasets based upon the RF model, the t-test model and the correlation model, taking the subjects' assigned group (ASD or TD) as output variables. Eighty percent of subjects were randomly assigned to a training dataset and the remaining 20% of subjects to a test dataset. The trained model's area under the curve (AUC) was then calculated for the test dataset as an evaluation metric. This process was repeated 1,000 times in order to obtain a rigorous evaluation while suppressing any bias which could have been caused by favorable data splits.

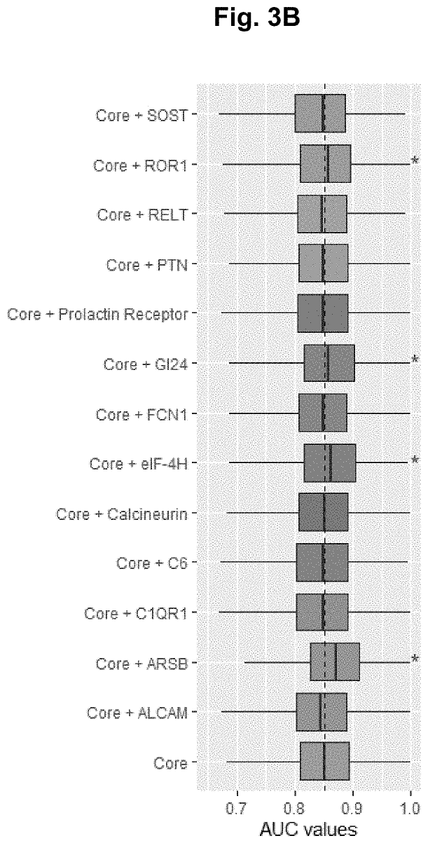

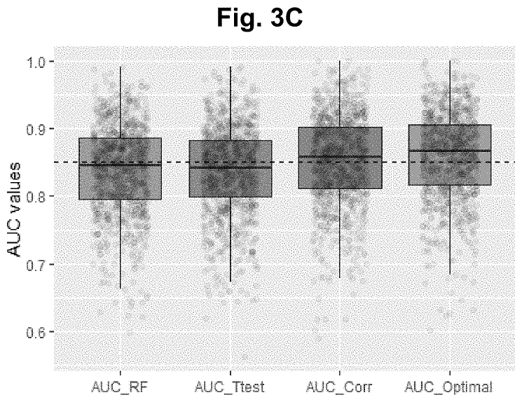

In order to optimize the predictive power of the biomarker panel, it was first sought whether there was any protein overlap among the three methods (FIG. 3A). Highlighted are the 5 core proteins that were common to all three methods. Each of the additional 13 proteins were successively added, one at a time, to see if they increased the predictive value of the AUC using logistic regression (FIG. 3B). Four additional proteins: receptor tyrosine kinase-like orphan receptor 1 [ROR1], platelet receptor Gl24 [GI24], eukaryotic translation initiation factor 4H [elF-4H], and arylsulfatase B [ARSB], increased the AUC when each was added to the core proteins. The AUC for the 10 proteins identified by each model was: RF=0.839±0.066, t-test=0.837±0.066 and correlation=0.853±0.066. Combining the 5 core proteins with the additional 4 proteins resulted in an AUC=0.860±0.064, with a sensitivity=0.8333±0.118, and specificity=0.846±0.118 (FIG. 3C), and represents the 9 optimal proteins (AUC Optimal).

A pathway enrichment analysis was performed for the optimal proteins. Entrez Gene identifiers corresponding to the optimal proteins were fed to a limma::goana function in R. From its gene ontology results, the top-20 biological process pathways were extracted and reported. The top-20 biological processes from pathway enrichment analysis are shown in Table 4. The 9 optimal proteins have pathway significance related to a number of processes associated with immune function in ASD.

TABLE 4
Top-20 GO terms from pathway enrichment analysis.

| | | Total | Genes | | |
|---|---|---|---|---|---|
| GO ID | GO Term | genes | in list | Related protein | p-value |
| G0: 0051239 | regulation of multicellular | 3144 | 7 | GI24, suPAR, | 5.90E−05 |
| | organismal process | | | ARSB, MAPK14, | |
| | | | | EPHB2, ROR1, | |
| GO: 0044093 | positive regulation of | 1636 | 5 | GI24, suPAR, | 3.30E−04 |
| | molecular function | | | MAPK14, EPHB2, | |
| | | | | ROR1 | |
| GO: 0061580 | colon epithelial cell | 1 | 1 | ARSB | 4.45E−04 |
| | migration | | | | |
| GO: 2000565 | negative regulation of | 1 | 1 | GI24 | 4.45E−04 |
| | CD8-positive, alpha- | | | | |
| | beta T cell proliferation | | | | |
| GO: 1904782 | negative regulation of | 1 | 1 | EPHB2 | 4.45E−04 |
| | NMDA glutamate | | | | |
| | receptor activity | | | | |
| GO: 0106028 | neuron projection | 1 | 1 | EPHB2 | 4.45E−04 |
| | retraction | | | | |
| GO: 0120158 | positive regulation | 1 | 1 | GI24 | 4.45E−04 |
| | of collagen catabolic | | | | |
| | process | | | | |
| GO: 0051240 | positive regulation of | 1750 | 5 | GI24, ARSB, | 4.53E−04 |
| | multicellular organismal | | | MAPK14, | |
| | process | | | EPHB2, IgD | |
| GO: 0045597 | positive regulation of cell | 962 | 4 | GI24, ARSB, | 5.31E−04 |
| | differentiation | | | MAPK14, EPHB2 | |
| GO: 0040011 | locomotion | 1835 | 5 | GI24, suPAR, | 5.66E−04 |
| | | | | ARSB, | |
| | | | | MAPK14, | |
| GO: 0052547 | regulation of peptidase | 398 | 3 | GI24, suPAR, | 5.83E−04 |
| | activity | | | MAPK14 | |
| GO: 0051389 | inactivation of MAPKK | 2 | 1 | EPHB2 | 8.91E−04 |
| | activity | | | | |
| GO: 0061582 | intestinal epithelial cell | 2 | 1 | ARSB | 8.91E−04 |
| | migration | | | | |
| GO: 1900450 | negative regulation of | 2 | 1 | EPHB2 | 8.91E−04 |
| | glutamate receptor | | | | |
| GO: 0038195 | urokinase plasminogen | 2 | 1 | SuPAR | 8.91E−04 |
| | activator signaling pathway | | | | |
| GO: 2000026 | regulation of multicellular | 2077 | 5 | GI24, ARSB, | 1.01E−03 |
| | organismal development | | | MAPK14, EPHB2, | |
| | | | | ROR1 | |
| GO: 0043312 | neutrophil degranulation | 485 | 3 | suPAR, ARSB, | 1.04E−03 |
| | | | | MAPK14 | |
| GO: 0002283 | neutrophil activation | 488 | 3 | suPAR, ARSB, | 1.05E−03 |
| | involved in immune | | | MAPK14 | |
| | resnonse | | | | |
| GO: 0042119 | neutrophil activation | 499 | 3 | suPAR, ARSB, | 1.13E−03 |
| | | | | MAPK14 | |
| GO: 0002446 | neutrophil mediated | 499 | 3 | suPAR, ARSB, | 1.13E−03 |
| | immunity | | | MAPK14 | |

To determine the accuracy of the SomaScan™ assay, duplicate blood samples were analyzed from 14 subjects (7 ASD and 7 TD). The 9 proteins selected for the optimal ASD biomarker panel exhibited an average of 6 to 13% variability between the duplicate assays.

Finally, to evaluate possible confounding factors, ethnicity, allergies, age, and medication use were analyzed as independent variables using t-tests or Spearman's rank correlation, as appropriate. To test the effect of ethnicity the data were split into two groups, white (n=73) and non-white (n=81) subjects. To test the effect of allergy, the data were split into two groups: patients with allergies (n=96) and patients without allergies (n=58). T-tests were then run to compare the two modified datasets for each of the core proteins. To test the effect of age, a Spearman's rank correlation was ran for each protein against the age distribution of subjects. To test the effect of psychiatric medications, the AUC values for the full data set (n=154) were compared with the AUC values of the dataset without the 10 subjects reporting medication usage (n=144).

Neither ethnicity nor a diagnosis of allergies had an effect on the mean protein counts of the optimal proteins as shown in Table 5.

TABLE 5
Analysis of the effect of ethnicity (t-test), season allergies (t-test), and age (Spearman
Rank correlation) on the 9 optimal ASD biomarker proteins.

| | | | Age | |
|---|---|---|---|---|
| | Ethnicity | Allergies | Correlation | |
| Core Protein | P Value | P Value | Coefficient | P Value |
| Derm | 0.0945599457 | 0.506182808 | −0.1716170 | 0.0333225559 |
| EPHB2 | 0.874995881 | 0.3372426933 | −0.1952733 | 0.0152265032 |
| suPAR | 0.0771914709 | 0.3967884756 | 0.09769581 | 0.2280561331 |
| IgD | 0.3710600063 | 0.1824142231 | −0.02575595 | 0.7511894219 |
| MAPK14 | 0.8177752333 | 0.940352121 | −0.1597256 | 0.0478479594 |
| ARSB | 0.5130854458 | 0.4461669899 | 0.07164907 | 0.3772184183 |
| eIF-4H | 0.5962435575 | 0.6179682879 | 0.01414712 | 0.8617558074 |
| GI24 | 0.9117334514 | 0.6633725258 | 0.06822360 | 0.4005076684 |
| ROR1 | 0.7357798354 | 0.702088237 | −0.1493244 | 0.0645523301 |

For age, all of the correlation coefficients were small (r=−0.17 to 0.098; Table 5), indicating there is no age effect on protein counts. The use of psychiatric medication did not significantly impact the AUC for the optimal proteins: the AUC for the total dataset was 0.8599, whereas the AUC for the dataset with the 10 subjects reporting medication use removed was 0.8440 (Table 6).

TABLE 6
Comparison of IL-8 and TSH levels in ASD and TD boys, and
correlation with ADOS total scores.

| | | T-test | | | |
|---|---|---|---|---|---|
| | | Mean | | Correlation with ADOS | |
| Group | Protein | (normalized) | p-value | Coefficient | p-value |
| ASD | IL-8 | −0.1763751 | 0.002 | 0.1808 | 0.0248 |
| TD | IL-8 | −0.2291225 | | | |
| ASD | TSH | 0.06342079 | 0.007 | −0.1841 | 0.0223 |
| TD | TSH | 0.1956321 | | | |

The data provided in Example 1 disclosed nine proteins that were identified based upon a novel combination of machine learning methods with random forest analysis, t-test analysis, and correlation analysis with ADOS total scores that produced an accurate identification of ASD in boys. Five of the proteins, IgD, suPAR, MAPK14, EPHB2, and DERM, were present in all three analyses and were considered core proteins in the panel. Four proteins providing additive power were combined with the 5 core proteins, and together, the 9 proteins resulted in an AUC of 86% (sensitivity 83%; specificity 84%). These proteins have pathway significance related to a number of processes, including negative regulation of CD8-positive, alpha-beta T cell proliferation, immune response, neuron projection retraction, MAPK14 activity, and glutamate receptor signaling. Ethnicity, age, and use of psychiatric medication did not impact the protein counts for the biomarker panel. This novel set of proteins provided herein can be—individually or in combination—efficacious blood-based biomarkers for the early identification of ASD in boys, particularly since behavioral and developmental assessments are not easily administered in very young children.

### Example 2

The methods and discovery of a core set of ASD biomarker proteins described in Example 1 can be used to diagnose ASD in a subject who has not yet been diagnosed with ASD. In brief, a sample of blood serum is collected from a subject suspected of having ASD. The serum sample is subjected to SomaLogic SOMAScan™ platform using a methodology similar to that disclosed in Shubin et al., Sci Data 6, 314 (2019), the disclosure of which is incorporated herein in its entirety.

The resulting protein abundance data is then normalized. In brief, the data are log 10-transformed and the log 10-transformed data are concatenated with the control group (existing data of 154 male pediatrics established in Example 1). Then, with z-transformation, the new subject's z-score for each protein is calculated. Z-transformation compares protein expression in the serum sample (experimental sample) with that of control group protein expression. For each core protein identified in Example 1 (i.e., Derm, EPHB2, suPAR, IgD, MAPK14, ARSB, eIF-4H, GI24, ROR1) a z-score is calculated. The z-score for each protein is the normalized protein quantity for the new subject. Now, the normalized values of 9 proposed biomarker proteins are directly input to a logistic regression-based prediction model.

The logistic regression-based prediction model returns an output score ranging between 0 and 1. If the output score is below 0.5, the subject suspected of having ASD who was submitted to testing is predicted as an ASD-like pediatric. If the output score is greater 0.5, the subject suspected of having ASD who was submitted to testing is predicted as a normal-like pediatric.

1. A method of identifying enhanced risk of Autism spectrum disorder (ASD) comprising:
(a) determining expression levels of a panel of ASD markers in a biological sample, wherein the panel of ASD markers comprise a group of ASD markers consisting essentially of: immunoglobulin D (IgD), soluble urokinase-type plasminogen activator receptor (suPAR), mitogen-activated protein kinase 14 (MAPK14), ephrin type-B receptor 2 (EPHB2), dermatopontin (DERM); and
(b) identifying an enhanced risk of ASD in a subject having a biological sample in which the expression levels of the panel of ASD markers is differentially expressed compared to expression levels of the ASD markers in a control biological sample.

2. The method of claim 1, wherein the panel of ASD markers comprise a group of ASD markers consisting essentially of: receptor tyrosine kinase-like orphan receptor 1 (ROR1), platelet receptor Gl24 (GI24), eukaryotic translation initiation factor 4H (elF-4H), arylsulfatase B (ARSB), immunoglobulin D (IgD), soluble urokinase-type plasminogen activator receptor (suPAR), mitogen-activated protein kinase 14 (MAPK14), ephrin type-B receptor 2 (EPHB2), and dermatopontin (DERM).

3. (canceled)

4. The method of claim 1, wherein a differential expression level comprises a higher or lower expression level of the ASD marker in the subject sample compared to the control sample.

5. The method of claim 4, wherein a lower expression level in a subject sample of MAPK14, IgD, DERM, EPHB2, eIF-4H, and ROR1 compared to the control sample and an increased expression level in the subject sample of suPAR, ARSB and GI24 compared to the control sample identifies an enhanced risk of ASD in a subject.

6-7. (canceled)

8. The method of claim 1, wherein the subject is a human.

9. The method of claim 8, wherein the human is a human child younger than 16 years old.

10. The method of claim 9, wherein the human child is a human male child.

11. The method of claim 1, wherein the biological sample comprises a whole blood sample, a blood serum sample, a blood plasma sample, or any combination thereof.

12-22. (canceled)

23. The method of claim 1, wherein the control biological sample is a biological sample from a control subject determined not to be at risk of ASD from assessment of
non-genetic factors selected from the group consisting of Autism Diagnostic Observation Schedule (ADOS), Autism Diagnostic Interview-Revised (ADI-R), the Adaptive Behavior Assessment System-Second Edition (ABAS-II), and/or Diagnostic and Statistical Manual of Mental Disorders (DSM-5) Autism Diagnostic Criteria.

24. The method of claim 1 further comprising treating
(a) the subject identified as having an enhanced risk of ASD by
(b) administering to the subject at least one treatment for ASD.

25. The method of claim 24, wherein the at least one treatment for ASD comprises applied behavior analysis, assistive technology, social skills training, speech therapy, dietary treatment, pharmaceutical treatment, or any combination thereof.

26-30. (canceled)

31. The method of claim 1, wherein the level of the ASD markers is determined by one or more of the following: Western blotting, enzyme-linked immunosorbent assay (ELISA), mass spectrometry, HPLC, flow cytometry, fluorescence-activated cell sorting (FACS), liquid chromatography-mass spectrometry (LC/MS), immunoelectrophoresis, translation complex profile sequencing (TCP-seq), protein microarray, protein chip, capture arrays, reverse phase protein microarray (RPPA), two-dimensional gel electrophoresis or (2D-PAGE), functional protein microarrays, electrospray ionization (ESI), and matrix-assisted laser desorption/ionization (MALDI).

32. A kit for determining levels of at least one marker of Autism spectrum disorder (ASD) in a subject, the kit comprising a means for determining protein levels of a panel of ASD markers comprising any combination of ASD markers selected from the group consisting essentially of immunoglobulin D (IgD), soluble urokinase-type plasminogen activator receptor (suPAR), mitogen-activated protein kinase 14 (MAPK14), ephrin type-B receptor 2 (EPHB2), dermatopontin (DERM), receptor tyrosine kinase-like orphan receptor 1 (ROR1), platelet receptor Gl24 (GI24), eukaryotic translation initiation factor 4H (elF-4H), and arylsulfatase B (ARSB).

33. The kit of claim 32, wherein the means for determining protein levels comprises a set of aptamers, each of which is for detecting one of the ASD markers in the ASD panel, and wherein the set of aptamers collectively detects the protein levels of the markers in the ASD panel.

34. The kit of claim 33, wherein the set of aptamers is attached to a solid support.

35-37. (canceled)

38. A method for determining enhanced risk of Autistic spectrum disorder (ASD) comprising:
determining an expression level of a panel of ASD markers in a subject biological sample, wherein the panel of ASD markers consists essentially of:
immunoglobulin D (IgD), soluble urokinase-type plasminogen activator receptor (suPAR), mitogen-activated protein kinase 14 (MAPK14), ephrin type-B receptor 2 (EPHB2), dermatopontin (DERM), receptor tyrosine kinase-like orphan receptor 1 (ROR1), platelet receptor Gl24 (GI24), eukaryotic translation initiation factor 4H (elF-4H), arylsulfatase B (ARSB); and
identifying an enhanced risk of ASD in a subject having a subject biological sample having protein expression levels of ASD markers that are differentially expressed compared to expression levels of the ASD markers in a control biological sample,
wherein an enhanced risk of ASD is identified for the subject having
an expression level of IgD, MAPK14, EPHB2 and DERM that is decreased compared to an expression level of IgD, MAPK14, EPHB2 and DERM from the control biological sample; and
an expression level of suPAR that is increased compared to an expression level of suPAR from the control biological sample.

39. The method of claim 38 wherein an enhanced risk of ASD from a subject biological sample is identified where the expression levels of eIF-4H and ROR1 of the subject biological sample are decreased compared to the expression levels of eIF-4H and ROR1 in the control biological sample.

40. The method of claim 38 wherein an enhanced risk of ASD from a subject biological sample is identified where the expression level of GI24 and ARSB of the subject biological sample is increased compared to the expression level of GI24 and ARSB in the control biological sample.
